(19) United States
(12) Patent Application Publication
(10) Pub. No.: US 2025/0336531 A1
(43) Pub. Date: Oct. 30, 2025

(54) SYSTEM AND METHOD FOR GENERATING AND IMPLEMENTING A DIAGNOSTIC MODULE DATABASE FOR CHARACTERIZING PATIENT DIAGNOSES

(71) Applicant: Regard Technologies, Inc., Los Angeles, CA (US)

(72) Inventors: Daniel Lee Tryon, New York City, NY (US); Francisco Alvarez, New York, NY (US); Kevin Hannigan, Hermosa, CA (US)

(21) Appl. No.: 19/192,851

(22) Filed: Apr. 29, 2025

Related U.S. Application Data
(60) Provisional application No. 63/640,114, filed on Apr. 29, 2024.

(51) Int. Cl. G16H 50/20; A61B 5/00; G06F 40/289; G06F 40/40; G16H 10/60

(52) U.S. Cl. CPC G16H 50/20; A61B 5/742; G06F 40/289; G16H 10/60; G06F 40/40

(57) ABSTRACT

One variation of a method includes, during a first time period: initializing a diagnosis module corresponding to a diagnosis; extracting language signals, corresponding to the diagnosis, from a medical resource; interpreting a set of diagnosis indicators supporting the diagnosis based on the language signals; populating the diagnosis module with the set of diagnosis indicators; and storing the diagnosis module in a diagnostic module database. This variation of the method also includes, during a second time period succeeding the first time period: receiving confirmation of an encounter between a patient and a provider; predicting a positive diagnosis for the diagnosis exhibited by the patient based on a set of patient data extracted from a health record associated with the patient; generating a prompt to review the positive diagnosis for the patient; and serving the prompt to the provider.

### CROSS-REFERENCE TO RELATED APPLICATIONS

This application claims the benefit of U.S. Provisional Application No. 63/640,114, filed on 29 Apr. 2024, which is incorporated in its entirety by this reference.

### TECHNICAL FIELD

This invention relates generally to the field of health care and, more specifically, to a new and useful system and method for predicting diagnoses of patients in the field of health care.

### BRIEF DESCRIPTION OF THE FIGURES

FIG. 1 is a flowchart representation of a method;

FIGS. 2A and 2B are flowchart representations of one variation of the method;

FIG. 3 is a flowchart representation of one variation of the method;

FIGS. 4A and 4B are flowchart representations of one variation of the method; and

FIG. 5 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

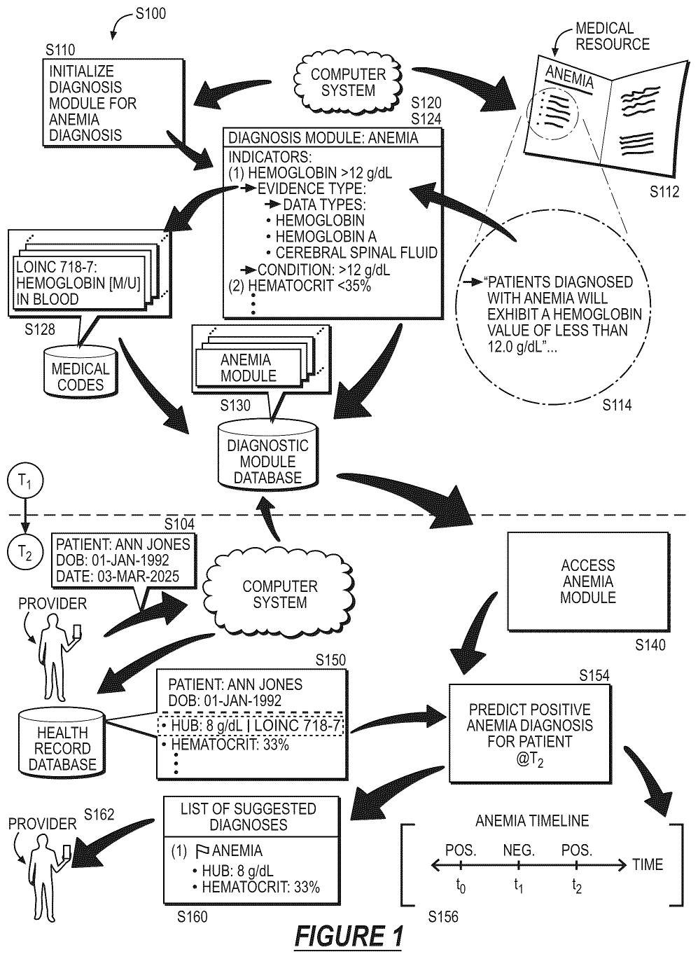

As shown in FIGS. 1, 2A, 2B, and 5, a method S100 includes, during a first time period: initializing a first diagnosis module, in a population of diagnosis modules, for a first diagnosis in a population of diagnoses in Block S110; accessing a medical resource specifying diagnosis indicators supporting the first diagnosis in Block S112; extracting a first cluster of language signals, corresponding to the first diagnosis, from the medical resource in Block S114; isolating a first set of language signals, in the first cluster of language signals, defining explicit diagnosis criteria for the first diagnosis; and transforming the first set of language signals into an explicit diagnosis indicator supporting the first diagnosis in Block S120. The method S100 further includes, during the first time period: isolating a second set of language signals, in the first cluster of language signals, implying diagnosis criteria for the first diagnosis; interpreting an interpreted diagnosis indicator supporting the first diagnosis based on the second set of language signals in Block S120; and storing the first diagnosis module, including the explicit diagnosis indicator and the interpreted diagnosis indicator, in a diagnostic module database including the population of diagnosis modules in Block S130.

The method S100 also includes, during a second time period, succeeding the first time period, during a first encounter between a first patient and a first provider: receiving the first diagnosis for the first patient from the first provider via a provider portal executing on a computing device accessed by the first provider in Block S106; accessing the first diagnosis module, in the population of diagnosis modules of the diagnostic module database, defining the explicit diagnosis indicator and the interpreted diagnosis indicator in Block S140; scanning a first health record, in a population of health records and associated with the first patient, for units of patient data corresponding to the explicit diagnosis indicator and the interpreted diagnosis indicator; and extracting a first set of patient data, from the first health record, corresponding to the explicit diagnosis indicator and supporting the first diagnosis for the first patient in Block S150.

The method S100 further includes, during the second time period: in response to correspondence between the first set of patient data and the first diagnosis module, generating a first notification including the first set of patient data and a first prompt to review the first set of patient data and the first diagnosis for insertion into a provider note generated for the first encounter in Block S164; and serving the first notification to the first provider via the provider portal in Block S162.

### 1.1 Variation: Predicting Diagnoses

As shown in FIGS. 1, 3, and 5, one variation of the method S100 includes, during a first time period: initializing a first diagnosis module, in a population of diagnosis modules, for a first diagnosis in a population of diagnoses in Block S110; accessing a medical resource specifying diagnosis indicators supporting the first diagnosis in Block S112; extracting a first cluster of language signals, corresponding to the first diagnosis, from the medical resource in Block S114; and interpreting a set of diagnosis indicators supporting the first diagnosis based on the first cluster of language signals in Block S120.

This variation of the method S100 further includes, during the first time period: retrieving a set of medical codes, in a population of medical codes, linked to the set of diagnosis indicators in Block S128; and storing the first diagnosis module, including the set of diagnosis indicators linked to the set of medical codes, in a diagnostic module database including the population of diagnosis modules in Block S130.

This variation of the method S100 also includes, during a second time period succeeding the first time period: receiving confirmation of a first encounter between a first patient and a first provider via a provider portal executing on a computing device accessed by the first provider in Block S104; accessing the first diagnosis module, in the population of diagnosis modules of the diagnostic module database, defining the set of diagnosis indicators in Block S140; scanning a health record, in a population of health records and associated with the first patient, for units of patient data corresponding to the set of diagnosis indicators; extracting a first set of patient data, from the health record, corresponding to the set of diagnosis indicators in Block S150; and predicting the first diagnosis for the first patient during the first encounter based on correspondence between the first set of patient data and the set of diagnosis indicators, defined in the first diagnosis module, that indicate presence of the first diagnosis in Block S154.

This variation of the method S100 further includes, during the second time period: generating a first notification including a first prompt to review the positive diagnosis for including in a provider note generated for the first encounter in Block S160; and serving the first notification to the first provider via the provider portal in Block S162.

### 1.2 Variation: Patient Data Extraction Based on Medical Codes

As shown in FIGS. 1, 4A, 4B, and 5, one variation of the method S100 includes, during a first time period: initializing a diagnosis module, in a population of diagnosis modules, for a diagnosis in a population of diagnoses in Block S110; accessing a medical resource specifying diagnosis indicators supporting the diagnosis in Block S112; extracting a cluster of language signals, corresponding to the diagnosis, from the medical resource in Block S114; and interpreting a set of diagnosis indicators supporting the diagnosis based on the cluster of language signals, each diagnosis indicator in the set of diagnosis indicators defining an evidence type and a value of the evidence type and indicating presence of the diagnosis for a patient in Block S120.

This variation of the method S100 further includes, during the first time period, for a first diagnosis indicator, in the set of diagnosis indicators, defining a first evidence type and a first value of the first evidence type: accessing a first set of data types, in a population of defined data types, corresponding to the first evidence type; retrieving a first set of medical codes, in a population of medical codes, linked to the first set of data types in Block S128; and storing the first diagnosis indicator, linked to the first set of medical codes, in the diagnosis module in Block S124. This variation of the method S100 also includes, during the first time period, storing the diagnosis module in a diagnostic module database including the population of diagnosis modules in Block S130.

This variation of the method S100 also includes, during a second time period succeeding the first time period: receiving confirmation of an encounter between a patient and a provider via a provider portal executing on a computing device accessed by the provider in Block S104; accessing the diagnosis module, in the population of diagnosis modules of the diagnostic module database, defining the set of diagnosis indicators in Block S140; and scanning a health record, in a population of health records, associated with the patient for units of patient data corresponding to the set of diagnosis indicators. This variation of the method S100 further includes, during the second time period, extracting a first unit of patient data from the health record, the first unit of patient data: corresponding to a first medical code, in the first set of medical codes, and a first data type in the first set of data types corresponding to the first evidence type; and satisfying the first value of the first evidence type in Block S150.

This variation of the method S100 also includes, during the second time period: predicting the diagnosis for the patient during the encounter based on the first unit of patient data satisfying the first value of the first evidence type, defined in the diagnosis module, that indicates applicability of the diagnosis in Block S154; appending a diagnosis timeline, representing timeseries diagnosis data exhibited by the patient over time, with the diagnosis in Block S156; generating a notification including a prompt to review the positive diagnosis for including in a provider note generated for the encounter in Block S160; and serving the notification to the provider via the provider portal in Block S162.

### 1.2 Variation: Note Categorization+Selective Note Surfacing

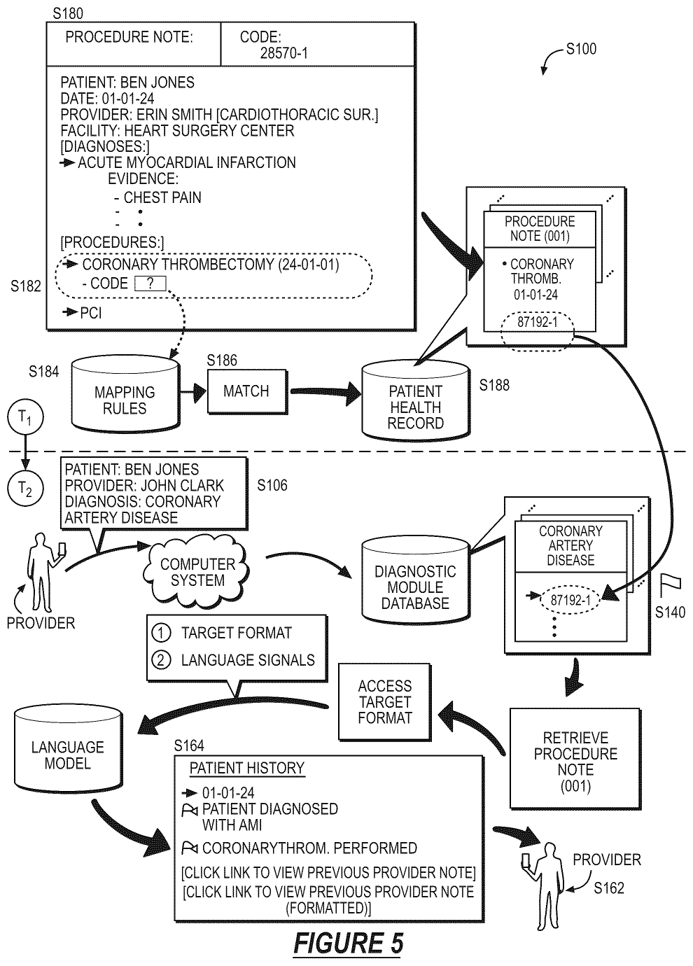

As shown in FIGS. 1 and 5, one variation of the method S100 includes, during a first time period: accessing an unstructured provider note previously generated by a first provider for a first encounter between a patient and the first provider in Block S180; extracting a cluster of language signals from the unstructured provider note; identifying a set of language signals—in the cluster of language signals—corresponding to a unit of patient data and omitting a medical code in a population of medical codes in Block S182; accessing a set of mapping rules associating language signals corresponding to units of patient data with medical codes in Block S184; and predicting a confidence score for the first set of language signals corresponding to a first medical code—in the population of medical codes—based on a correlation between the first set of language signals and criteria defined for the first medical code in the set of mapping rules in Block S186.

This variation of the method S100 further includes, during the first time period, in response to the confidence score exceeding a threshold score: associating the first medical code with the first set of language signals and the first unit of patient data; and storing the first unit of patient data in association with the first medical code in a health record—in a population of health records—associated with the patient in Block S188.

This variation of the method S100 further includes, during a second time period succeeding the first time period, during a second encounter between the patient and a second provider: receiving identification of a first diagnosis, in a population of diagnoses, for the patient from the provider via a provider portal executing on a computing device accessed by the second provider in Block S106; and accessing a diagnosis module, in the population of diagnosis modules of the diagnostic module database, defining a set of diagnosis indicators in Block S140.

This variation of the method S100 further includes, during the second time period, for a first diagnosis indicator—defining an evidence type and a value of the evidence type that supports the first diagnosis—in the set of diagnosis indicators: retrieving a set of medical codes—corresponding to a set of medical data types—linked to the first diagnosis indicator and including the first medical code in Block S128; and scanning the health record associated with the patient for units of patient data corresponding to the set of medical codes. This variation of the method S100 further includes, during the second time period, in response to extracting the first unit of patient data—linked to the first medical code—in the health record associated with the patient: transforming the first unit of patient data into a first unit of formatted patient data in a target data format; retrieving the unstructured provider note including the first unit of patient data; and transforming the unstructured provider note into a structured provider note in a target note format. This variation of the method S100 also includes, during the second time period, in Block S162, generating a notification including: the first unit of formatted patient data in the target data format; a prompt to review the first unit of formatted patient data for insertion into a provider note generated for the encounter; a first link configured to trigger rendering of a first window loaded with the unstructured provider note; and a second link configured to trigger rendering of a second window loaded with the structured provider note. This variation of the method S100 further includes, during the second time period, serving the notification to the provider via the provider portal in Block S162.

### 1.4 Variation: Manual Diagnosis Module Generation Via Builder Portal

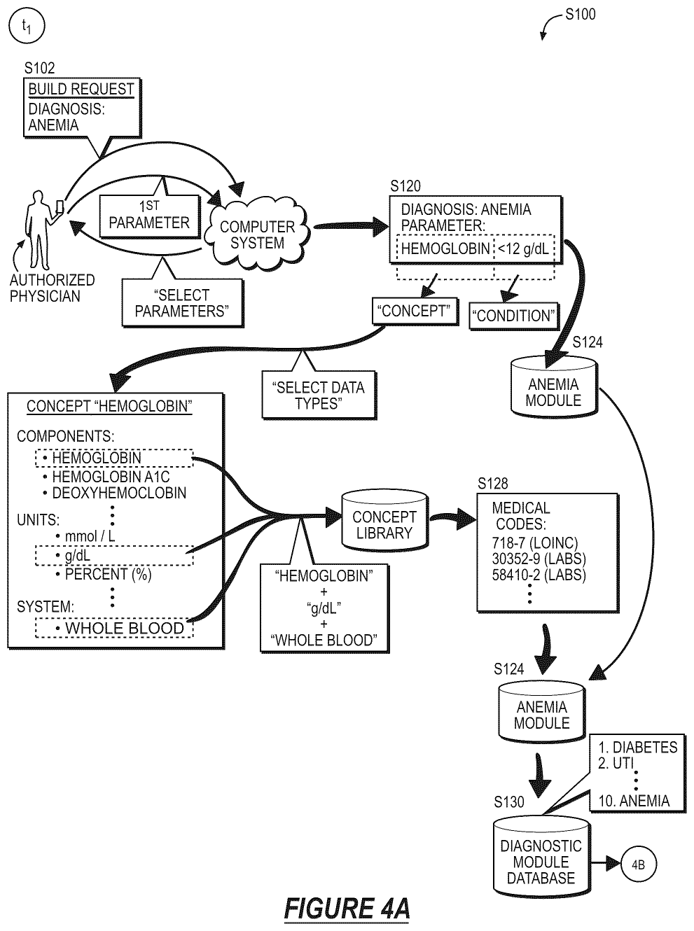

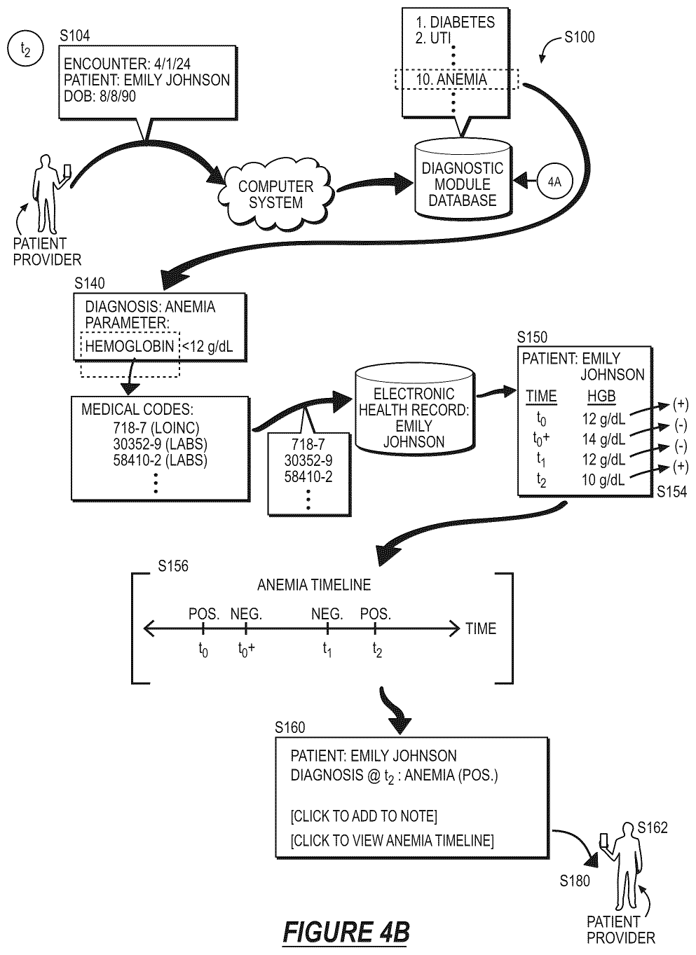

As shown in FIGS. 4A and 4B, one variation of the method S100 includes, during a first time period, receiving a build request—specifying a diagnosis in a population of diagnoses—from a provider via an instance of a builder portal executing on a computing device accessed by the provider in Block S102. This variation of the method S100 further includes, during the first time period, in response to receiving the build request: initializing a first diagnosis module, in a population of modules, for the diagnosis in Block S110; presenting a diagnosis survey—including a request to define a set of diagnosis indicators supporting the diagnosis—to the provider within the builder portal; receiving a first set of diagnosis indicators—supporting the diagnosis—from the provider, each diagnosis indicator, in the first set of diagnosis indicators, defining an evidence type and a value—defined for the evidence type—indicating applicability of the particular diagnosis to a patient in Block S120; and populating the diagnosis module with the first set of diagnosis indicators corresponding to the particular diagnosis in Block S124.

This variation of the also includes, during the first time period, for a first diagnosis indicator, in the first set of diagnosis indicators, defining a first evidence type: prompting the provider to select a set of data types, in a population of defined data types, corresponding to the first evidence type; accessing an evidence type library linking a population of medical codes (e.g., CPT, LOINC, RxNorm) to the population of defined data types; based on a first set of data types—selected by the provider within the builder portal—and the evidence type library, retrieving a first set of medical codes, in the population of medical codes, linked to the first set of data types in Block S128; associating a first medical code group—including the first set of medical codes—with the first evidence type in the first diagnosis module generated for the particular diagnosis; and storing the first diagnosis module, in the population of modules, in a diagnostic module database including the population of diagnosis modules in Block S130.

This variation of the method S100 further includes, at a first time during a second time period succeeding the first time period: receiving confirmation of an encounter between a patient and a provider (e.g., the patient's physician) via an instance of a provider portal executing on a computing device accessed by the provider in Block S106; accessing a set of patient data extracted from an electronic health record—affiliated with the patient; accessing the first diagnosis module, in the population of modules of the diagnostic module database, defining the first set of diagnosis indicators in Block S140; for the first diagnosis indicator, in the set of diagnosis indicators, defined in the first diagnosis module, extracting a first subset of patient data, from the set of patient data, associated with the first set of medical codes in Block S150; and, in response to the first subset of patient data corresponding to the first diagnosis indicator, assigning a true value—in a set of values derived for the first set of diagnosis indicators—to the first diagnosis indicator.

This variation of the method S100 further includes, during the second time period: based on the set of values, predicting a positive diagnosis for the first diagnosis exhibited by the patient at the first time in Block S154; appending a diagnosis timeline—representing timeseries diagnosis data for the first diagnosis exhibited by the patient over time—with the positive diagnosis at the first time in Block S156; generating a notification including a prompt to review the positive diagnosis for including in a provider note generated for the encounter; appending the notification with a link to view the diagnosis timeline for the particular diagnosis in Block S164; and transmitting the notification to the provider via the provider portal in Block S162.

### 2. Applications

Blocks of the method S100 can be executed by a computer system—interfacing with a builder portal—accessed by a provider—to: receive a build request from the provider; initialize a diagnosis module, in a population of modules, defining a particular diagnosis in a population of diagnoses (e.g., medical diagnoses) in response to receiving the build request; populate the diagnosis module with a set of diagnosis indicators corresponding to the particular diagnosis, based on selections entered by the provider within the builder portal; and store this module in a diagnostic module database linking the population of diagnoses to types of evidence supporting each diagnosis in the population of diagnoses.

Furthermore, the computer system can interface with a provider portal—accessed by a patient's medical provider (e.g., the patient's physician) to: receive identification of a patient from the patient's medical provider via the provider portal; retrieve a health record (e.g., an electronic health record)—including demographic data (e.g., age, sex, geographic location), timestamped test data (e.g., historical and/or current vitals, lab results, imaging results), lifestyle data (e.g., stress level, activity level), medication data, etc.—corresponding to the patient; access the diagnostic module database including the population of modules; scan the patient's health record for patient data corresponding to diagnoses indicators defined in each of these modules; and selectively predict a particular diagnosis (or diagnoses) and/or present evidence—supporting a diagnosis provided by the patient's medical provider—for the patient based on correlations between patient data (e.g., a blood pressure reading, a resting heart rate, a white blood cell count, a body temperature, an oxygen saturation) derived from the patient's health record and diagnoses indicators defined by a particular module, in the population of modules, corresponding to the particular diagnosis.

Generally, the computer system can host or interface with a builder portal (e.g., a native application or web application) executing on a computing device (e.g., a mobile device, a computer) accessed by a provider to selectively generate a diagnosis module for a particular diagnosis. The computer system can then store this module in a population of modules—corresponding to a population of diagnoses—in the diagnostic module database. Later, the computer system can access this diagnostic module database to selectively: predict diagnoses for patients based on patient data and diagnosis elements defined in each module in the population of modules; and/or surface patient data supporting diagnoses of patients provided by providers.

In one implementation, the computer system can store and implement a diagnostic module database loaded with a population of modules—representative of a population of diagnoses—each module, in the population of modules, defining: a particular diagnosis in the population of diagnoses; a set of diagnosis indicators (i.e., units and/or combinations of evidence supporting the particular diagnosis)—such as derived from timeseries health data including vital data (e.g., body temperature, pulse rate, respiration rate, blood pressure, weight), lab data (e.g., white blood cell count, culture presence), image data (e.g., X-ray data, CT scan data), demographic information (e.g., age, sex, geographic location), lifestyle data (e.g., activity level, sleep level, stress level), medication data, etc.—that support the particular diagnosis; and/or a set of treatment pathways (e.g., medications, procedures, lifestyle changes, activities) associated with treatment of the diagnosis. In this implementation, the computer system can initialize a new module, in the population of modules, for a particular diagnosis in response to receiving a build request. In particular, the computer system can host or interface with a builder portal—executing on a computing device accessed by a provider—to: receive a build request for a new diagnosis; and, in response to receiving the build request, initialize a new module for the new diagnosis and present a builder interface—such as including one or more prompts to enter various selections corresponding to the new diagnosis—to the provider within the builder portal.

For example, the computer system can prompt the provider to define and/or select one or more diagnoses indicators—each diagnoses indicator defining an evidence type and a value defined for the evidence type—that support the new diagnosis. In one example, the provider may: enter a diagnosis of “anemia” for the new module; define a first diagnosis indicator of “hemoglobin less than 12 units per volume”; and define a second diagnoses indicator of “mean corpuscular volume less than 80 volumetric units.” The computer system can then store this diagnosis and the first and second diagnoses indicators in the new module generated for the “anemia” diagnosis.

Furthermore, for each evidence type defined in a diagnosis indicator, the computer system can prompt the provider—within the builder portal—to select a set of concept elements (e.g., defined data types) corresponding to the evidence type, such as for this particular diagnosis. In particular, in the preceding example, the computer system can prompt the provider to select a set of concept element types to link to a first evidence type of “hemoglobin”—defined for the first diagnosis indicator of “hemoglobin less than 12 units per volume”—for a diagnosis of anemia. For example, the computer system can prompt the provider to select: one or more types of hemoglobin, from a set of hemoglobin types, such as including “hemoglobin,” “hemoglobin A1c,” “hemoglobin A,” hemoglobin A2,” etc.; and/or one or more sources of hemoglobin, such as including “serum plasma,” “cerebral spinal fluid,” etc.

The computer system can then: receive a set of concept elements selected by the provider via the builder portal; access an evidence type library linking each concept element, in a population of concept elements, to one or more medical codes in a population of predefined medical codes—such as including LOINC codes defined for a set of labs, procedures, vitals, imaging reports, etc., RxNorm codes defined for a set of medications, SNOMED codes defined for a set of findings, ICD10 codes defined for a set of conditions—associated with medical systems; and, based on the set of concept elements selected by the provider, extract a set of medical codes, from the population of medical codes, corresponding to the set of concept elements. The computer system can then automatically associate this set of medical codes with the first evidence type of “hemoglobin” defined for the “anemia” diagnosis.

### 2.1 Automatic Diagnosis Module Generation

In one application, the computer system can execute Blocks of the method S100 to automatically: initialize a diagnosis module for a particular diagnosis; ingest and/or access a medical resource, such as a medical textbook, a clinical guideline (e.g., a Centers for Disease Control and Prevention (CDC) Guideline), or a peer-reviewed journal article; extract content (e.g., symptoms, evidence types, time windows) related to the diagnosis from the medical resource; transform this content into diagnosis indicators supporting the diagnosis; store each diagnosis indicator in the diagnosis module corresponding to the diagnosis; render the diagnosis module for an authorized physician (or an “operator”) to review (e.g., via the provider portal); and prompt the authorized physician to review and confirm (e.g., accept and/or approve) the diagnosis indicators supporting the diagnosis.

In particular, the computer system can: implement a language model to extract natural language signals, related to diagnosis indicators of a particular diagnosis, from the medical resource; transform these natural language signals into structured diagnosis indicators defining indicator parameters (e.g., evidence types, values, and/or time windows); prompt the provider (i.e., an authorized physician) to review and approve these diagnosis indicators and select or adjust the derived and/or interpreted indicator parameters; and store a parameterized (i.e., fixed), provider-approved diagnosis module, defining the diagnosis indicators, within the diagnostic module database. Therefore, the computer system can: parse medical resources to rapidly generate a parametrized diagnosis module from natural language medical content; render this diagnosis module for review by an authorized physician; and store the approved diagnosis module within the diagnostic module database for later access during an encounter with a patient to predict the diagnosis for the patient.

In particular, upon generation of a diagnosis module, the computer system can selectively prompt an authorized physician to review and confirm the diagnosis module for insertion into the diagnostic module database. For example, the computer system can prompt a hematologist to review a newly-generated anemia module, such as when the hematologist accesses an instance of the provider portal, based on a match between the provider's clinical discipline (i.e., hematology) and the diagnosis. In another example, the computer system can prompt a provider to review the diagnosis module during a patient encounter when the patient exhibits indicators supporting the diagnosis (i.e., upon implementation of the diagnosis module). In yet another example, the computer system can prompt an operator (e.g., an authorized physician licensed to review and confirm diagnosis modules in multiple specialties) to review the diagnosis module inferring one or more diagnosis indicators with low confidence (e.g., implied thresholds or time windows derived from vague language signals).

Accordingly, the computer system can: automatically generate the diagnosis module in response to gaining access to clinical content relevant to the diagnosis; and queue this newly-generated diagnosis module for review and/or selectively prompt an authorized physician to review the diagnosis module, such as based on timing, clinical discipline alignment, or low-confidence inference. Therefore, the computer system can opportunistically request provider review and confirmation of diagnosis modules without disrupting the provider's workflow during a patient encounter and/or interfering with patient care.

Then, during a patient encounter between a patient and the patient's medical provider (e.g., a physician), the computer system can: query a secure database for patient data associated with the patient and/or relevant to this encounter; scan all contents of the patient's health record, such as including all recent and historical health data collected for this patient over time; access the “anemia” module, in the population of modules of the diagnostic module database, generated for a diagnosis of anemia; and, in response to identifying the first diagnosis indicator of “hemoglobin less than 12 units per volume,” automatically extract all patient data—from the patient's health record—linked to the set of medical codes defined for the evidence type of “hemoglobin” in the “anemia” module. The computer system can then leverage patient data corresponding to the set of medical codes—such as including a “hemoglobin from serum plasma” level of 10 units per volume—to evaluate whether patient data matches the value (e.g., “less than 12 units per volume) defined for the evidence type of “hemoglobin”. In this example, in response to the “hemoglobin from serum plasma” level of 10 units per volume falling below “12 units per volume” as defined in the first diagnosis indicator, the computer system can assign a “true” value to the first diagnosis indicator. The computer system can then selectively surface patient data—corresponding to the first diagnosis indicator of the “anemia” module for a diagnosis of anemia—to the patient's medical provider for further review of whether the patient exhibits a positive anemia diagnosis. The computer system can therefore: surface key evidence—supporting a diagnosis supplied by the provider and assigned a “true” value by the diagnostic module database—to the provider within the provider portal; and/or selectively surface a predicted and/or possible diagnosis for the patient—supplemented with key evidence supporting the possible diagnosis and assigned a “true” value by the diagnostic module database—to the provider within the provider portal. Accordingly, during a patient encounter, the computer system can access these parameterized, provider-approved diagnosis modules to selectively predict diagnoses and/or support provider-suggested diagnoses for a patient based on fixed diagnostic criteria (e.g., without reliance on a diagnosis prediction model).

### 3. Provider Portal+Patient Data

Generally, the computer system can host or interface with a provider portal (e.g., a native application or web application) executing on a computing device (e.g., a mobile device, a computer) accessed by a provider to selectively guide and/or support the provider in generating a provider note (hereinafter a “note”) for a patient encounter.

In one implementation, the computer system can integrate with a cloud platform employed by a health care system (e.g., a hospital or hospital system, a clinic, a health care group)—such as an electronic health record (or “EHR”) platform or database—in order to access health records (e.g., EHRs) containing historical and/or current health information of patients affiliated with the health care system. For example, the computer system can integrate with an EHR platform employed by the health care system and therefore access a population of health records corresponding to a population of patients affiliated with the health care system, each health record, in the population of health records, containing timeseries patient data captured for a patient, in the population of patients, over time and including: a list of timestamped encounters for this patient at a facility affiliated with the health care system; a series of timestamped notes—each note corresponding to a timestamped encounter in the list of timestamped encounters—previously generated for this patient; a list of timestamped medications prescribed to and/or employed by the patient (e.g., historically and/or currently); a series of timestamped vital data (e.g., heart rate, respiratory rate, body temperature, oxygen saturation) recorded for the patient; a series of timestamped test results—including labs and/or images—obtained for the patient; a list of timestamped medical diagnoses or symptoms exhibited by the patient; etc.

### 4. Diagnostic Module Database

Generally, the computer system can access a diagnostic module database linking each diagnosis, in a population of diagnoses, to a set of supporting evidence (or “diagnosis indicators”) that support the diagnosis and/or to a set of treatment pathways corresponding to the diagnosis. For example, the computer system can access a diagnostic module database linking: a first diagnosis to a first set of diagnosis indicators—such as derived from patient vitals, labs, images, etc.—and a first set of treatment pathways (e.g., medications, procedures, lifestyle changes) associated with the first diagnosis; a second diagnosis to a second set of diagnosis indicators and a second set of treatment pathways associated with the second diagnosis; a third diagnosis to a third set of diagnosis indicators and a third set of treatment pathways associated with the third diagnosis; etc. The computer system can therefore access a diagnostic module database that links each diagnosis, in the population of diagnoses, to diagnosis indicators (i.e., evidence) and/or combinations of diagnosis indicators that support the diagnosis and treatment pathways configured to treat and/or mitigate the diagnosis, and thereby represents key information (e.g., diagnoses, diagnosis indicators, treatment pathways) required for generation and/or acceptance of notes.

In one implementation, the diagnostic module database can include a population of modules, each module, in the population of modules, corresponding to a particular diagnosis in a population of diagnoses. In particular, each module (e.g., a data container), in the population of modules, can define: a diagnosis; a set of diagnosis indicators associated with (e.g., supporting) the diagnosis; and a set of treatment pathways associated with the diagnosis.

For example, the diagnostic module database can include a population of modules including a first diagnosis module defining: a first diagnosis of pneumonia; a first set of diagnosis indicators—such as including a target body temperature, a target white blood cell count, a target oxygen saturation, a target chest image (e.g., a target chest x-ray, a target chest CT), a target procalcitonin level, etc.—associated with pneumonia; and a first set of treatment pathways—such as including a set of antibiotics and/or a set of medical procedures (e.g., intubation, extubation)—associated with treatment and/or mitigation of pneumonia. The population of modules can further include a second diagnosis module defining: a second diagnosis of acute, systolic, congestive heart failure; a second set of diagnosis indicators—such as including a target left ventricular ejection fraction (or “LVEF”), a target body weight, a target B-type natriuretic peptide (or “BNP”) level, a target chest x-ray, etc.—associated with acute, systolic, congestive heart failure; and a second set of treatment pathways—such as including a set of medications—associated with treatment and/or mitigation of acute, systolic, congestive heart failure. Furthermore, the population of modules can include a third module defining: a third diagnosis of Type II Diabetes Mellitus (or “adult onset diabetes”); a third set of diagnosis indicators—such as including a target glucose level, a target hemoglobin A1C (or “HbA1c”) level, a target diabetic neuropathy level (e.g., specified by the patient)—associated with Type II Diabetes Mellitus; and a third set of treatment pathways—such as including a set of insulin medications—associated with treatment and/or mitigation of Type II Diabetes Mellitus.

The computer system can therefore leverage this diagnostic module database—in combination with historical patient data (e.g., medical records, lab results, provider notes, conditions, procedures, medications, observations, reports, encounters) retrieved for a particular patient—to: selectively present patient data supporting a diagnosis supplied by a provider via the provider portal; and/or selectively suggest diagnoses to the provider for a particular patient. The computer system can therefore access patient data—such as during and/or at a start of an encounter between the patient and the provider—corresponding to data types defined in the diagnostic module database and feed this patient data into corresponding modules of the diagnostic module database accordingly.

### 5. Diagnosis Module Generation

Blocks of the method S100 recite: initializing a diagnosis module, in a population of diagnosis modules, for a diagnosis in a population of diagnoses in Block Silo; accessing a medical resource specifying diagnosis indicators supporting the diagnosis in Block S112; extracting a cluster of language signals, corresponding to the diagnosis, from the medical resource in Block S114; interpreting a set of diagnosis indicators supporting the diagnosis based on the cluster of language signals in Block S120; populating the diagnosis module with the set of diagnosis indicators supporting the diagnosis in Block S124; and storing the diagnosis module in a diagnostic module database linking the population of diagnoses to diagnoses indicators supporting each diagnosis in the population of diagnoses in Block S130.

Generally, as shown in FIG. 1, the computer system can: ingest and/or access a medical resource, such as a medical textbook, a clinical guideline (e.g., a Centers for Disease Control and Prevention (CDC) Guideline), or a peer-reviewed journal article; extract content (e.g., symptoms, evidence types, time windows) related to a particular diagnosis from the medical resource; transform this content into diagnosis indicators supporting the diagnosis; store each diagnosis indicator in a diagnosis module corresponding to the particular diagnosis; render the diagnosis module for a provider to review (e.g., via the provider portal); and prompt the provider to review and confirm (e.g., accept and/or approve) the diagnosis indicators supporting the diagnosis.

The computer system can then store this module (e.g., an approved module) in a population of modules—corresponding to a population of diagnoses—in the diagnostic module database. Later, the computer system can access this diagnostic module database to selectively: predict diagnoses for patients based on patient data and diagnosis elements defined in each module in the population of modules; and/or surface patient data supporting diagnoses of patients suggested by a provider.

### 6. Content Extraction from Medical Resources

Generally, for a particular diagnosis, the computer system can: scan a medical resource for content related to the diagnosis; and transform language signals, related to diagnosis indicators supporting the diagnosis, into diagnosis indicators that support the diagnosis.

In one implementation, the computer system can: initialize a diagnosis module for a diagnosis (e.g., bacterial pneumonia) in the population of diagnoses; access a medical resource (e.g., an infectious disease textbook) specifying diagnosis indicators supporting the diagnosis; and extract a cluster of language signals, corresponding to the diagnosis, from the medical resource. In particular, the computer system can scan the medical resource for language signals containing diagnostic criteria, symptom descriptions, clinical measurements, lab values, and/or interrelated diagnoses. The computer system can then transform these language signals into a diagnosis indicator supporting the diagnosis and including: an evidence type (e.g., a white blood cell count); a value (e.g., greater than 11,000 cells per microliter) indicating that the diagnosis indicator is satisfied for the patient; and a time window (e.g., within 48 hours prior to diagnosis).

### 6.1 Explicitly Supported Diagnosis Indicators

Blocks of the method S100 recite: extracting a cluster of language signals, corresponding to the diagnosis, from the medical resource in Block S114; defining an explicit diagnosis indicator supporting the diagnosis based on a set of language signals, in the cluster of language signals, explicitly supporting the first diagnosis indicator in Block S120; and storing the explicit diagnosis indicator in the diagnosis module corresponding to the diagnosis in Block S124. Generally, for a particular diagnosis, the computer system can: scan a medical resource for content related to the diagnosis; extract language signals that explicitly define a particular diagnosis indicator; transform these language signals into a diagnosis indicator (i.e., an explicit diagnosis indicator) supporting the diagnosis; and store this diagnosis indicator in a diagnosis module corresponding to the diagnosis. In particular, the computer system can scan the medical resource for language signals containing explicit descriptions of evidence types, values, and/or time windows corresponding to diagnosis indicators supporting the diagnosis.

In one example, for a diagnosis module for an anemia diagnosis, the computer system can: extract a cluster of language signals from a medical resource; and identify a set of language signals, in the cluster of language signals, such as “Anemia is diagnosed when a blood test shows a hemoglobin value of less than 12.0 g/dL in female patients.” The computer system can then define a diagnosis indicator supporting the anemia diagnosis and defining: an evidence type, such as a hemoglobin laboratory test result; and a value, such as a hemoglobin value less than 12.0 g/dL. Thus, the computer system can transform explicit clinical language into structured diagnostic criteria by directly defining a diagnosis indicator based on recognized evidence types and thresholds.

### 6.2 Interpreted Diagnosis Indicators

Blocks of the method S100 recite: extracting a cluster of language signals, corresponding to the diagnosis, from the medical resource in Block S114; interpreting an interpreted diagnosis indicator supporting the diagnosis based on a set of language signals, in the cluster of language signals, implying the interpreted diagnosis indicator in Block S120; and storing the interpreted diagnosis indicator in the diagnosis module corresponding to the diagnosis in Block S130. Generally, for a particular diagnosis, the computer system can: scan a medical resource for content related to the diagnosis; extract language signals that imply a particular diagnosis indicator; transform these language signals into a diagnosis indicator (i.e., an interpreted diagnosis indicator) supporting the diagnosis; and store this diagnosis indicator in a diagnosis module corresponding to the diagnosis. In one example, for a diagnosis module for an anemia diagnosis, the computer system can: extract a cluster of language signals from a medical resource; and identify a set of language signals, in the cluster of language signals, such as “patient with anemia will often describe symptoms of feeling faint.” The computer system can then interpret a diagnosis indicator supporting the anemia diagnosis and defining: an evidence type, such as “fatigue;”; and a value, such as “yes.”

In one variation, the computer system can define and/or interpret various parameters (e.g., evidence type, time window) of a diagnosis indicator based on explicit and/or explicit support for these parameters. For example, for a particular diagnosis, the computer system can: extract a cluster of language signals, corresponding to the diagnosis, from the medical resource; and isolate a set of language signals, in the cluster of language signals, that correspond to a diagnosis indicator supporting the diagnosis. In particular, in this example, computer system can: interpret an evidence type corresponding to the diagnosis indicator based on the set of language signals implying the evidence type; and interpret a threshold range corresponding to the evidence type based on the set of language signals implying the threshold range.

Alternatively, computer system can: define an evidence type corresponding to the diagnosis indicator based on the set of language signals (e.g., “anemia is diagnosed by evaluating hemoglobin levels”) explicitly supporting the evidence type; and interpret a threshold range corresponding to the evidence type based on the set of language signals (e.g., “a value below 12.0 g/dL is considered abnormal in females”) implying the threshold range. In another example, the computer system can: interpret an evidence type corresponding to the diagnosis indicator based on the set of language signals (e.g., “patients with anemia often report chronic fatigue”) implying the evidence type; and interpolate a time window based on the set of language signals (e.g., “symptoms typically develop over a period of several weeks”).

More specifically, the computer system can: isolate a first subset of language signals, in a set of language signals corresponding to a diagnosis indicator, including phrases referencing clinical observations of the interpreted diagnosis indicator; match the first subset of language signals to an evidence type, in the population of evidence types, based on correspondence between the first subset of language signals and criteria defined for the evidence type; isolate a second subset of language signals, in the set of language signals, including values proximal the evidence type in the medical resource; interpret a value of the evidence type, implied by the second subset of language signals, that indicates applicability of the diagnosis to patients; isolate a third subset of language signals, in the set of language signals, that imply timing of the evidence type; and interpret a time window of the evidence type implied by the third subset of language signals. Accordingly, the computer system can: define diagnosis indicator parameters directly from language explicitly specifying clinical criteria; and infer missing parameters based on the language signals. Thus, the computer system can rapidly generate structured, auditable, and clinically relevant diagnosis indicators from natural language medical content.

### 6.2.1 Threshold Confidence in Interpreted Diagnosis Indicators

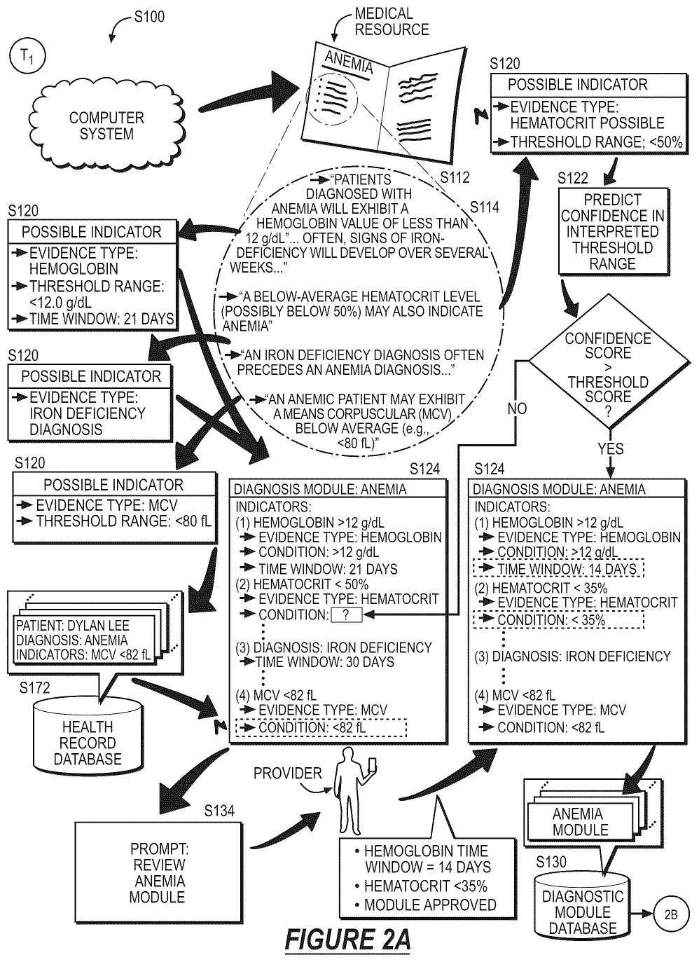

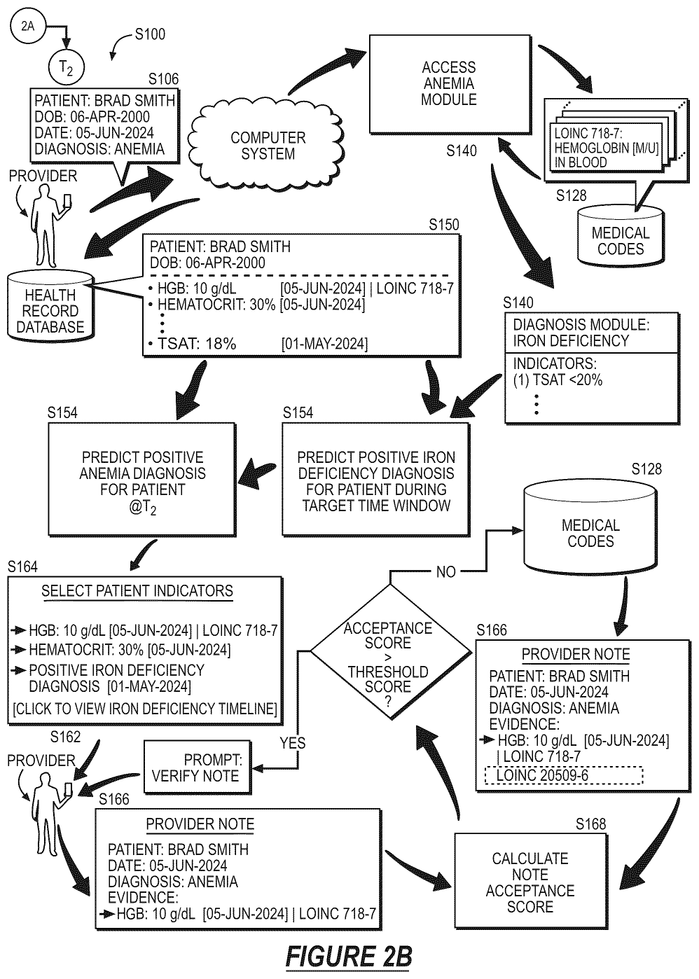

In one variation, as shown in FIG. 2A, Block S122 of the method S100 recites calculating a confidence score for the threshold range corresponding to the evidence type. In this variation, the computer system can: calculate a confidence score for an interpreted diagnosis indicator (or an interpreted diagnosis indicator parameter); and selectively retain or discard diagnosis indicators based on the confidence score. In one example, for a diagnosis module corresponding to a particular diagnosis, the computer system can: interpret a diagnosis indicator defining an evidence type (e.g., an explicitly supported evidence type); interpret a first threshold range (e.g., an implied threshold range) corresponding to the evidence type; and calculate a confidence score for the first threshold range corresponding to the evidence type. In response to the confidence score exceeding a threshold score, the computer system can store the diagnosis indicator, defining the evidence type and the first threshold range, in the diagnosis module. Alternatively, in response to the confidence score falling below the threshold score, the computer system can: discard the first threshold range; and prompt the provider to select a second threshold range corresponding to the evidence type. Thus, the computer system can maintain the accuracy and reliability of diagnosis indicators by filtering out low-confidence diagnosis indicators.

### 6.2.2 Interpreted Diagnosis Indicators Based on Historical Records

In one variation, as shown in FIG. 2A, Blocks of the method S100 recite: interpreting a diagnosis indicator, supporting a diagnosis, defining an evidence type and a first threshold range based on a set of language signals implying the evidence type and the first threshold range in Block S120; accessing a set of health records, in the population of health records, associated with positive diagnosis of the diagnosis in Block S172; deriving a second threshold range, different from the first threshold range, corresponding to the evidence type based on threshold ranges associated with positive diagnosis of the first diagnosis represented in the set of health records; and calculating a third threshold range based on a union of the first threshold range and the second threshold range.

In this variation, the computer system can: access historical records associated with positive diagnosis of a particular diagnosis; and leverage these historical records to validate or refine diagnosis indicators (or parameters of diagnosis indicators), such as based on statistical patterns represented in patient data associated with positive (e.g., confirmed) diagnoses.

In one example, for a diagnosis module for a particular diagnosis, the computer system can: interpret a diagnosis indicator defining an evidence type (e.g., an implied evidence type); and interpret a first threshold range (e.g., an implied threshold range) corresponding to the evidence type. The computer system can then: access a set of health records, in the population of health records, associated with positive diagnosis of the diagnosis; derive a second threshold range, different from the first threshold range, corresponding to the evidence type based on threshold ranges, corresponding to the evidence type and associated with positive diagnosis of the diagnosis, represented in the set of health records; and calculate a third threshold range based on a union of the first threshold range and the second threshold range. The computer system can then prompt the provider to review the diagnosis indicator, defining the evidence type and the third threshold range, for insertion into the diagnosis module. Thus, the computer system can validate or refine inferred diagnosis indicators by augmenting language-derived thresholds with empirically derived values from historical records associated with positive diagnosis of the diagnosis.

### 7. Diagnosis Module Review

Block S134 of the method S100 recites generating a notification including a prompt to review the diagnosis module for insertion into the diagnostic module database. Generally, upon generation of a diagnosis module, the computer system can render the diagnosis module within the builder portal for the provider to review. In particular, the computer system can render each diagnosis indicator (i.e., criteria required for diagnosing a patient with the diagnosis) defined in the diagnosis module within the builder portal. More specifically, for each diagnosis indicator, the computer system can: render the diagnosis indicator within the builder portal; generate an audit trail representing the language signals extracted from the medical resource to derive the diagnosis indicator; and render a link, adjacent the diagnosis indicator, configured to trigger rendering of a window loaded with the audit trail.

In one implementation, upon generating a diagnosis module, the computer system can: generate a prompt to review the diagnosis module for insertion into the diagnostic module database; and serve the notification to the provider (e.g., via the provider portal). In particular, the computer system can prompt the provider to: review each diagnosis indicator (e.g., interpreted or explicitly supported diagnosis indicator) extracted from the medical resource; adjust parameters (e.g., time windows, threshold ranges) of these diagnosis indicators; define parameters (e.g., a time window absent in the medical resource) of these diagnosis indicators; and/or define additional diagnosis indicators supporting the diagnosis. The provider—accessing the diagnosis module via the builder portal—may then: selectively adjust parameters of each diagnosis indicator extracted from the medical resource; and/or selectively enter one or more additional diagnosis indicators supporting to the diagnosis.

In one example, the computer system: implements methods and techniques described above to interpret a diagnosis indicator defining an evidence type and a first time window of the evidence type; and renders the diagnosis indicator for the provider to review via the builder portal. In response to receiving selection of a second time window, different from the first time window, from the provider, the computer system: updates the diagnosis indicator to define the evidence type and the second time window; and stores the diagnosis indicator in the diagnosis module.

Additionally, upon reviewing each diagnosis indicator defined in the diagnosis module and/or selecting additional diagnosis indicators, the provider can confirm (e.g., approve) the diagnosis module for the diagnosis. In response to receiving confirmation of the diagnosis module from the provider, the computer system can store the diagnosis module in a diagnostic module database. Therefore, the computer system can: parse medical resources to rapidly generate a parametrized diagnosis module from natural language medical content; render this diagnosis module for review by a provider; and store the approved diagnosis module within the diagnostic module database for later access during an encounter with a patient to predict the diagnosis for the patient.

### 7.1 Opportunistic Diagnosis Module Review

In one variation, the computer system can selectively prompt the provider to review the diagnosis module for insertion into the diagnostic module database upon implementation of the diagnosis module. In one example, the computer system can implement methods and techniques described above to generate a diagnosis module corresponding to a particular diagnosis and defining a first diagnosis indicator and a second diagnosis indicator. Then, during an encounter with a patient, the computer system can implement methods and techniques described above to extract a unit of patient data corresponding to the first diagnosis indicator and supporting the particular diagnosis for the patient. In particular, in this example, in response to the unit of patient data supporting the diagnosis for the patient and in response to absence of a representative range of values for the second diagnosis indicator in the diagnosis module, the computer system can: generate a notification to select the representative range of values of the second diagnosis indicator; and serve the second notification to the provider via the provider portal. In response to receiving a range of values from the provider, the computer system can then append the range of values, associated with the second diagnosis indicator, to the diagnosis module.

In another variation, the computer system can selectively prompt the provider to review the diagnosis module for insertion into the diagnostic module database upon inferring one or more diagnosis indicators with low confidence (e.g., implied thresholds or time windows derived from vague language signals). For example, in the preceding example, the computer system can: estimate a first range of values of the second diagnosis indicator (e.g., implied by the medical resource); calculate a confidence score for the first range of values of the second diagnosis indicator; and write the second diagnosis indicator, associated with the first range of values and the confidence score, to the diagnosis module. Then, during the encounter with a patient, in response to the unit of patient data supporting the diagnosis for the patient and in response to the confidence score for the second diagnosis indicator falling below a threshold confidence score, the computer system can: generate a notification to select a representative range of values of the second evidence type; and serve the second notification to the provider via the provider portal. In response to receiving a second range of values from the provider, the computer system can update the first range of values, in the diagnosis module, according to the second range of values.

In another variation, the computer system can selectively prompt the provider to review the diagnosis module for insertion into the diagnostic module database based on correspondence between the diagnosis and a clinical discipline of the provider. For example, in the preceding example, the computer system can: access a clinical discipline (e.g., hematology) of the provider; and, in response to the clinical discipline of the provider matching a clinical discipline of the diagnosis module (e.g., an anemia module), the computer system can implement methods and techniques described above to prompt the provider to review and confirm the diagnosis module. Therefore, the computer system can opportunistically request provider review and confirmation of diagnosis modules without disrupting the provider's workflow during a patient encounter and/or interfering with patient care.

### 8. Evidence Type Library+Medical Codes

Blocks of the method S100 recite: defining a diagnosis indicator defining an evidence type in a population of evidence types in Block S120; retrieving a set of medical codes, in a population of medical codes, linked to the evidence type in Block S128; and storing the set of medical codes in association with the diagnosis indicator in the diagnosis module in Block S124. Generally, the computer system can generate and/or update an evidence type library linking each evidence type—in a population of evidence types defined in the evidence type library—to a set of data types corresponding to the evidence type. Furthermore, the computer system can associate each data type with a set of standardized codes (hereinafter “medical codes”), such as including CPT codes defined for a set of procedures, LOINC codes defined for a set of labs, procedures, vitals, imaging reports, etc., RxNorm codes defined for a set of medications, SNOMED codes defined for a set of findings, ICD10 codes defines for a set of conditions, etc. The computer system can therefore link an evidence type to a set of (predefined) medical codes corresponding to the set of data types. The computer system can then leverage the evidence type library to selectively access patient data—such as from a patient's electronic health record—corresponding to the set of medical codes when evaluating a particular diagnoses indicator of a diagnosis module defining the corresponding evidence type.

In particular, in one implementation, the computer system can: access a population of medical codes—including CPT codes, LOINC codes, RxNorm codes, SNOMED codes, ICD10 codes, etc.—defined by a set of medical systems; leverage descriptions linked to each medical code, in the population of medical codes, to define a population of elements—such as including medical terms related to methods, systems, components, etc. understood by providers—linked to the population of medical codes. The computer system can then store this population of elements—linked to corresponding medical codes, in the population of medical codes, in an evidence type library.

Then, when generating a new module, in the population of modules, the computer system can define a diagnosis indicator—including an evidence type and/or a value of the evidence type—for a diagnosis associated with the new module. The computer system can access a set of elements, from the population of elements, associated with this evidence type for this particular diagnosis. In particular, computer system can access a set of elements—such as including methods, systems, components, etc. implemented and/or referenced in patient health records—that may be relevant in evaluating the diagnosis indicator defined for the diagnoses.

The computer system can then automatically translate a combination of elements into a set of medical codes corresponding to the combination of elements based on the evidence type library. The computer system can then automatically associate this set of medical codes with the new module for the diagnosis.

In one implementation, upon generating a diagnosis module for a diagnosis, the computer system can automatically retrieve the set of medical codes linked to the set of diagnosis indicators. In particular, for a diagnosis indicator defined in the diagnosis module, the computer system can: access a first evidence type corresponding to the diagnosis indicator; retrieve a first set of medical codes linked to the first evidence type; and store the first set of medical codes in association with the diagnosis indicator in the diagnosis module. Additionally, the computer system can implement methods and techniques described above to receive selection of a second evidence type corresponding to the diagnosis indicator from the provider (i.e., during review of the diagnosis module). In this variation, the computer system can then: retrieve a second set of medical codes linked to the second evidence type; and store the second set of medical codes in association with the diagnosis indicator in the diagnosis module.

In another variation, the computer system can retrieve the set of medical codes in response to receiving confirmation of the diagnosis module for the diagnosis from the provider. Alternatively, the computer system can retrieve the set of medical codes in response to receiving a trigger (e.g., receiving the diagnosis for a patient) to implement the diagnosis module. Thus, the computer system can withhold retrieval of the set of medical codes pending provider approval or implementation of the diagnosis module to reduce computational resources prior to module finalization.

In one example, the computer system can: initialize a diagnosis module for a diagnosis of “pancreatitis;” and define a first diagnosis indicator of “imaging report indicative of pancreatitis.” The computer system can then access a set of evidence types corresponding to the first diagnosis indicator of “imaging report indicative of pancreatitis.” For example, the computer system access: one or more imaging methods from a set of imaging methods, such as including “ultrasound,” “ultrasound doppler,” “ultrasound A scan,” “CT scan,” etc.; one or more body systems from a set of body systems, such as including “abdomen,” “abdomen+pelvis,” “abdomen+pelvis+lower extremity bilateral,” etc.; and/or one or more components from a set of components, such as including “guidance for aspiration,” “guidance for aspiration+placement of drainage tube,” “multisection,” “multisection-limited,” “multisection 3D post processing,” etc.

In the preceding example, the computer system can then automatically link a combination of evidence types—such as including the one or more methods, body systems, and/or components—to a particular set of medical codes, in a population of medical codes, associated with the combination of evidence types. For example, the computer system can: access a first medical code corresponding to a combination of “CT scan,” “abdomen,” and “guidance for percutaneous biopsy”; access a second medical code corresponding to a combination of “CT scan,” “abdomen+pelvis,” and “guidance for percutaneous biopsy”; access a third medical code corresponding to a combination of “CT scan,” “abdomen,” and “multisection”; access a fourth medical code corresponding to a combination of “CT scan,” “abdomen+pelvis,” and “multisection”; etc. The computer system can thus automatically link this set of medical codes—such as including the first, second, third, and fourth medical codes—to the first diagnosis indicator of “imaging report indicative of pancreatitis” defined for the “pancreatitis” module. The computer system can then store this diagnosis and the first diagnosis indicator—including the combination of evidence types and corresponding set of medical codes—in the new module generated for the “pancreatitis” diagnosis. Later, when implementing the diagnostic module database, in response to a diagnosis of pancreatitis, the computer system can automatically retrieve any patient data—from the patient's electronic health record—corresponding to this set of medical codes.

The computer system can therefore define particular types of data required for evaluating a diagnosis indicator defined in a diagnosis module for a particular diagnosis, such as in medical terms understood by the provider. The computer system can then leverage the evidence type library—linking evidence types to a population of medical codes—to automatically translate these terms and/or types of data into corresponding medical codes, without requiring the provider to specify these medical codes.

In another example, the computer system can: initialize a diagnosis module for a diagnosis of “anemia;” and define a first diagnosis indicator of “hemoglobin less than 12 units per volume (e.g., mmol/L or g/dL).” The computer system can then access a set of data types corresponding to a first evidence type of “hemoglobin” for a diagnosis of anemia. For example, the computer system access: one or more types of hemoglobin, from a set of hemoglobin data types, such as including “hemoglobin,” “hemoglobin A1c,” “hemoglobin A,” hemoglobin A2,” etc.; and/or one or more sources of hemoglobin, such as including “serum plasma,” “cerebral spinal fluid,” etc.

The computer system can then automatically link a combination of data types—such as including one or more hemoglobin data types and/or one or more sources of hemoglobin—to a particular set of medical codes, in a population of medical codes, associated with the combination of data types. In particular, in this example, for a set of data types including “hemoglobin” and “serum plasma,” the computer system can automatically select a set of medical codes, such as including: a first medical code corresponding to hemoglobin values derived from serum plasma drawn from a right arm of a patient; and a second medical code corresponding to hemoglobin values derived from serum plasma drawn from a left arm of a patient. Therefore, rather than require the provider to specify each medical code, in the population of medical codes, associated with hemoglobin values derived from serum plasma, the computer system can automatically link the set of data types—defined for the diagnosis indicator for a diagnosis of anemia—to the corresponding set of medical codes.

Later, when implementing the diagnostic module database, in response to a diagnosis of anemia, the computer system can automatically retrieve any patient data—from the patient's electronic health record—corresponding to this set of medical codes. Additionally or alternatively, in response to receiving a new set of patient data for the patient—including one or more medical codes in this set of medical codes—the computer system can automatically associate this new set of patient data with the diagnosis module, in the population of modules, generated for the “anemia” diagnosis.

### 9. Implementing the Diagnostic Module Database: Provider-Suggested Diagnosis

Blocks of the method S100 recite: receiving a diagnosis for a patient from a provider via a provider portal executing on a computing device accessed by a provider in Block S106; accessing a diagnosis module, in the population of modules of the diagnostic module database, corresponding to the diagnosis and defining a first diagnosis indicator in Block S140; extracting a set of patient data, from the health record, corresponding to the first diagnosis indicator and supporting the diagnosis for the patient in Block S150; generating a notification including the set of patient data and a prompt to review each unit of patient data, in the set of patient data, for insertion into a provider note generated for the encounter and indicating the diagnosis for the patient in Block S164; and serving the notification to the provider via the provider portal in Block S162.

Generally, the computer system can query a database for patient data for a particular patient in response to initiation of an encounter with this patient by a particular provider (e.g., within the provider portal). For example, the provider (e.g., the patient's physician) may access the provider portal and select a patient profile associated with the patient, thereby triggering initiation of an encounter between the patient and this provider. Then, in response to initiation of the encounter within the provider portal, the computer system can automatically generate and transmit a set of requests for patient data associated with the patient. In particular, in one example, in response to initiation of an encounter between the patient and the provider, the computer system receives a set of encounter data—corresponding to the particular encounter, patient, and/or provider—such as including: a patient identifier associated with the patient; a provider identifier associated with the provider; a timestamp corresponding to a start time of the encounter; and/or a data passcode (e.g., a dynamic passcode) configured to enable (temporary) access to patient data—stored in a secure database (e.g., remote from the computer system)—throughout a duration of the encounter. The computer system can then leverage the set of encounter data to query the secure database for patient data associated with the patient and/or relevant to this encounter.

In response to transmitting a request for patient data, the computer system can receive a set of patient data corresponding to the particular patient, such as for a particular encounter, a particular time period (e.g., 24 hours, one week, one year, ten years), and/or for a lifetime of the patient. The computer system can then insert the set of patient data into modules of the diagnostic module database to identify evidence—or units of patient data—that support diagnoses provided by the provider and/or to selectively suggest new diagnoses (e.g., no previously-provided by the provider).

For example, in response to receiving a first diagnosis for the patient from the provider via the provider portal, the computer system can: access a first diagnosis module, in the population of modules, of the diagnostic module database associated with the first diagnosis; extract a first diagnosis indicator—defining an evidence type and a value of the evidence type that supports the first diagnosis—in a set of diagnosis indicators corresponding to the first diagnosis; extract a set of medical codes—corresponding to a set of medical data types for the evidence type—associated with the first diagnosis indicator in the first diagnosis module; and automatically retrieve patient data, in the set of patient data, corresponding to this set of medical codes. Then, in response to a first unit of patient data, in the set of patient data (e.g., retrieved responsive to the request), corresponding to a first medical code in the set of medical codes—and thereby corresponding to the evidence type—the computer system can: input the first unit of patient data into the first diagnosis indicator to interpret whether the first unit of patient data satisfies the value defined for the evidence type; and selectively assign to the first diagnosis indicator a value of “true” or “false” based on whether the first unit of patient data satisfies the value.

In particular, in one example, the computer system can: access a diagnosis module, in the population of modules, corresponding to an “anemia” diagnosis; extract a diagnosis indicator defining an evidence type of “hemoglobin” and a value of “hemoglobin less than 12 g/dL”; extract a set of medical codes—corresponding to a set of medical data types, such as including “hemoglobin” and “serum plasma”—linked to the evidence type in this module; and scan the set of patient data for units of patient data corresponding to the set of medical codes. Then, in response to a first unit of patient data, in the set of patient data, corresponding to a first medical code, in the set of medical codes, the computer system can: extract a hemoglobin reading of 10 g/dL defined for the patient in the first unit of patient data; insert the hemoglobin reading of 10 g/dL into the first diagnosis indicator to interpret whether the first unit of patient data matches the diagnosis indicator of “hemoglobin less than 12 g/dL”; and, in response to the first unit of patient data matching the diagnosis indicator—such that the patient's hemoglobin reading is less than 12 g/dL—assign the diagnosis indicator a value of “true,” thereby supporting the diagnosis of “anemia.” In this example, the computer system can therefore: receive this “true” value assigned to the diagnosis indicator and output by the diagnostic module database; and then selectively surface this first unit of patient data to the provider within the provider portal to support the first diagnosis provided by the provider.

### 9.1 Acceptance Score+Provider Note Revisions

In one variation, Blocks of the method S100 recite: in response to receiving selection of a unit of patient data for insertion into the provider note from the provider via the provider portal, populating the provider note with the unit of patient data and a first medical code linked to the unit of patient data in Block S166; calculating a first acceptance score for the provider note based on the diagnosis, the unit of patient data, and the first medical code, the first acceptance score representing a likelihood of acceptance of the diagnosis for the patient during the encounter in Block S168; in response to the first acceptance score falling below a threshold score, interpreting a second medical code, in the population of medical codes, corresponding to the unit of patient data in Block S128; and populating the provider note with the second medical code linked to the unit of patient data in Block S166.

In particular, in this variation, the computer system can calculate an acceptance score for a provider note—indicating a particular diagnosis for a particular encounter with a patient—based on the particular diagnosis, units of patient data selected by the provider (and supporting the diagnosis for the patient) for insertion into the provider note, and medical codes linked to the units of patient data. For example, the computer system can calculate an acceptance score—representing a likelihood of acceptance of the provider note by an insurance company affiliated with the patient—for a provider note generated for an encounter with a patient. For example, the computer system can calculate an acceptance score represented by a percentage, a score between 1 and 10, a qualitative score (e.g., low, high, possible, probable), a binary score, etc. The computer system can compare the acceptance score to a threshold score—defined in the diagnostic module database—to calculate acceptance and/or rejection of a particular provider note.

In this variation, in response to calculating an acceptance score falling below a threshold score, the computer system can: withhold verification of the provider note and suggest selecting additional patient data, supporting the diagnosis, for insertion into the provider note; and/or populate the provider note with additional medical codes corresponding to the units of patient data selected by the provider. In particular, the computer system can interpret additional medical codes that are relevant to the selected unit of patient data (i.e., but not initially linked).

In one example, the computer system can: access a diagnosis module, in the population of modules, corresponding to an “anemia” diagnosis; extract a diagnosis indicator defining an evidence type of “hemoglobin” and a value of “hemoglobin less than 12 g/dL” and linked to a first medical code (e.g., LOINC 718-7 for Hemoglobin [Mass/volume] in Blood); scan the health record for units of patient data corresponding to the first medical code; and identify a set of patient data supporting the anemia diagnosis and including a first unit of patient data in the health record matching the diagnosis indicator. The computer system can then prompt the provider to select units of patient data, from the set of patient data, for insertion into the provider note. In response to receiving selection of the first unit of patient data for insertion into the provider note, the computer system can: calculate a first acceptance score for the provider note based on the first unit of patient data and the first medical code linked to the first unit of patient data; in response to the first acceptance score falling below a threshold score, interpret a second medical code (e.g., LOINC 20509-6 for Hemoglobin [Mass/volume] in Capillary Blood); and populate the provider note with the second medical code linked to the first unit of patient data.

The computer system can then calculate a second acceptance score for the provider note based on the first unit of patient data, the first medical code, and the second medical code. In response to the second acceptance score exceeding the threshold score, the computer system can then: compile a set of provider note data including the diagnosis, the units of patient data supporting the diagnosis and selected by a provider, and a treatment pathway, in a set of treatment pathways, selected by the provider; and transmit a notification—including a prompt to verify the provider note for transmitting to an insurance company affiliated with the patient—to the provider (e.g., via the provider portal). Therefore, the computer system can facilitate accurate and complete medical coding for each diagnosis, such as to reduce the risk of claim denial or documentation rejection by an insurance company affiliated with the patient.

### 9.2 Insufficient Evidence to Support Provider-Supplied Diagnosis

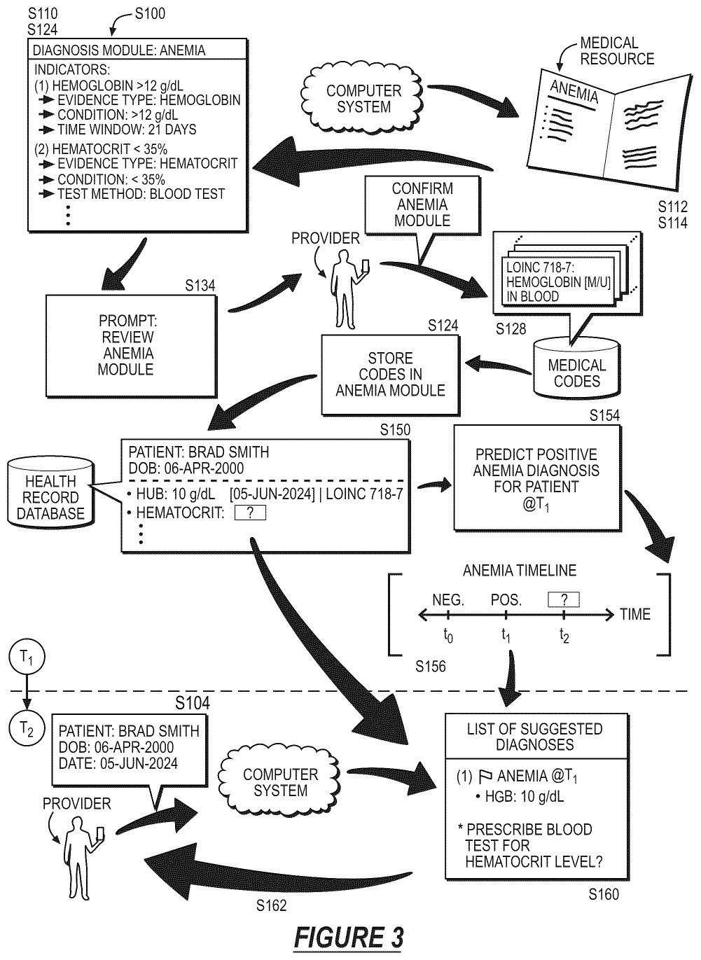

In one variation, as shown in FIG. 3, the computer system can suggest retrieval of additional patient data to supplement a diagnosis supplied by the provider, such as in response to confidence in note acceptance falling below a threshold confidence and/or the patient's health record missing a unit of patient data corresponding to an evidence type required to support a diagnosis. For example, the computer system can: generate a notification indicating insufficient evidence included in a note for a particular diagnosis and including a suggestion and/or prompt to execute a particular test—defined within a diagnosis module, in the diagnostic module database, corresponding to the diagnosis indicator—configured to retrieve additional health data that may support the particular diagnosis.

In this variation, the computer system can: receive a diagnosis from the provider (e.g., via the provider portal) for a patient, the diagnosis associated with the set of diagnosis indicators supporting the diagnosis including a first diagnosis indicator and a second diagnosis indicator; and extract a set of patient data—from the health record—including a first unit of patient data corresponding to the first target diagnosis indicator. The computer system can then generate and transmit a notification to the provider (e.g., via the provider portal) suggesting retrieval of additional health data for the patient in response to detecting absence of a second unit of patient data—in the health record—corresponding to the second target diagnosis indicator. In particular, the computer system can: access a medical test defined for the second diagnosis indicator, the medical test configured to yield a unit of patient data corresponding to the second diagnosis indicator; and transmit the notification to the provider (e.g., via the provider portal) indicating insufficient evidence for the diagnosis and including a suggestion to execute the medical test with the patient.

### 10. Implementing the Diagnostic Module Database: Predicted Diagnosis

Blocks of the method S100 recite: receiving confirmation of an encounter between a patient and a provider via a provider portal executing on a computing device accessed by the provider in Block S104; accessing a diagnosis module, in the population of modules of the diagnostic module database, defining a set of diagnosis indicators in Block S140; extracting a set of patient data, from the health record, corresponding to the set of diagnosis indicators and supporting the diagnosis for the patient in Block S150; predicting a positive diagnosis for the diagnosis exhibited by the patient during the encounter based on the set of patient data in Block S154; appending a diagnosis timeline, representing timeseries diagnosis data exhibited by the patient over time, with the diagnosis in Block S156; generating a notification including a prompt to review the positive diagnosis for including in a provider note generated for the encounter in Block S160; and serving the notification to the provider via the provider portal in Block S162.

In one implementation, the computer system can leverage the set of patient data to selectively predict and/or withhold prediction of a positive diagnosis for each diagnosis, in the population of diagnoses, defined in the diagnostic module database. In particular, in response to extracting the set of patient data from the health record, the computer system can automatically populate each module, in the population of modules, with a subset of patient data, in the set of patient data, corresponding to the diagnosis module based on a set of medical codes linked to both the subset of patient data and one or more diagnoses indicators defined by the diagnosis module.

In this implementation, the computer system can: access a first diagnosis module, in the population of modules of the diagnostic module database, defining the set of diagnosis indicators for a first diagnosis; scan a health record associated with the patient for units of patient data corresponding to the set of diagnosis indicators; extract a set of patient data, from the health record, corresponding to the set of diagnosis indicators and supporting the first diagnosis for the patient; and predict a positive diagnosis for the first diagnosis exhibited by the patient during the encounter based on the set of patient data. The computer system can then repeat this process for each diagnosis, in the population of diagnoses, to selectively present a set of diagnoses (or “suggested” diagnoses”) to the provider in (near) real-time during the encounter—such as within seconds of initiation of the encounter—for further review.

In response to flagging a diagnosis for further investigation by the provider, the computer system can selectively present the diagnosis to the provider via the provider portal. In particular, in response to flagging the diagnosis for further investigation, the computer system can: pair the diagnosis with units of patient data extracted from the set of patient data and supporting the diagnosis; and present the diagnosis and the units of patient data within the provider portal, such as below a set of existing diagnoses previously entered and/or selected by the provider.

Accordingly, for each diagnosis in the population of diagnoses, the computer system can: scan the patient's health record for units of patient data supporting the diagnosis for the patient; and selectively flag and/or withhold flagging of the diagnosis as a predicted diagnosis based on identification of the units of patient data—in the list of mapped patient data—supporting the diagnosis. Therefore, the computer system can generate a robust list of possible diagnoses for this patient and present this list of possible diagnoses—such as in a particular order based on urgency, confidence in diagnosis, relevancy to a current encounter, etc.—for review within the provider portal in (near) real-time.

### 11. Nested Diagnosis Modules

In one variation, as shown in FIG. 2B, the computer system can access a diagnosis indicator representing a nested diagnosis that supports another diagnosis, and access multiple diagnosis modules in parallel to evaluate related diagnostic criteria. In one example, the computer system can access a first diagnosis module, in the population of modules of the diagnostic module database, defining a first diagnosis indicator and a second diagnosis indicator for a first diagnosis (e.g., shingles). In particular, in this example, the first diagnosis indicator can define a first evidence type (e.g., a lab type) and a first value of the first evidence type. Additionally, the second diagnosis indicator can define a second diagnosis (e.g., chickenpox), in the population of diagnoses, correlated to the first diagnosis and exhibited by the patient within a time window (e.g., within the past 6 months).

The computer system can then: extract a first set of patient data, from the health record, corresponding to the first diagnosis indicator; access a second diagnosis module, in the population of modules of the diagnostic module database, for the second diagnosis, defining a second set of diagnosis indicators (e.g., positive viral culture, characteristic rash history, history of vesicular skin lesions—indicating prior chickenpox infection) supporting the second diagnosis; extract a second set of patient data, from the health record, corresponding to the second set of diagnosis indicators and supporting the second diagnosis for the patient during the time window; predict a positive diagnosis for the second diagnosis exhibited by the patient during the time window based on the second set of patient data; and append a diagnosis timeline, representing timeseries diagnosis data exhibited by the patient over time, with the second diagnosis. The computer system can then serve a list of patient data supporting the first diagnosis to the provider, the list of patient data including: the first set of patient data corresponding to the first diagnosis indicator; and the second set of patient data corresponding to the second diagnosis indicator. Additionally, the computer system can render the diagnosis timeline representing the second diagnosis exhibited by the first patient during the time window. Therefore, the computer system can: concurrently access interrelated modules to resolve nested or temporally linked diagnoses; and analyze longitudinal patient history to identify prior conditions that may support a current diagnosis.

### 12. Variation: Diagnosis Builder Tool

In one variation, as shown in FIGS. 4A and 4B, Blocks of the method S100 recite: receiving a build request—specifying a diagnosis in a population of diagnoses—from a provider via an instance of a builder portal executing on a computing device accessed by the provider in Block S102; initializing a first diagnosis module, in a population of modules, for the diagnosis in Block S110; receiving a first set of diagnosis indicators—supporting the diagnosis—from the provider in Block S120; and populating the diagnosis module with the first set of diagnosis indicators corresponding to the particular diagnosis in Block S124. In one variation, the computer system can host or interface with a builder portal (e.g., a native application or web application) executing on a computing device (e.g., a mobile device, a computer) accessed by a provider to selectively generate a diagnosis module for a particular diagnosis.

In this variation, the computer system can receive a build request to generate a new module via the builder portal. Then, in response to receiving the build request, the computer system can present a diagnosis survey—requesting a diagnosis and a set of diagnosis indicators—to the provider via the builder portal. For example, the computer system can present a diagnosis survey including: a prompt to define a diagnosis for the new module; and a prompt to define one or more diagnoses indicators—such as one or more criteria required for diagnosing a patient with the diagnosis—for the diagnosis. The provider—accessing the diagnosis survey via the builder portal—may then: enter a diagnosis (e.g., anemia, pneumonia, diabetes) for the new module, such as in a corresponding slot of the diagnosis survey; and selectively enter a set of diagnosis indicators corresponding to the diagnosis.

In one example, the provider may: enter a diagnosis of “urinary tract infection” for the new module; and define a first diagnosis indicator of “leukocyte esterase in urine exceeding ten units per volume.” In this example, when defining the first diagnosis indicator, the computer system can prompt the provider to select an evidence type and a value—for the evidence type—required to support the diagnosis for a patient. In particular, in this example, to define the first diagnosis indicator, the provider may select an evidence type of “leukocyte esterase in urine” and a value of “exceeding ten units per volume.” The computer system can then store this diagnosis and the first diagnosis indicator in the new module generated for the “urinary tract infection” diagnosis. Additionally or alternatively, in another example, the provider may: enter a diagnosis of “anemia” for the new module; define a first diagnosis indicator of “hemoglobin less than 12 units per volume (e.g., mmol/L or g/dL)”; and define a second diagnoses indicator of “mean corpuscular volume less than 80 volumetric units (e.g., 10⁻¹⁵; fl or μm³).” The computer system can then store this diagnosis and the first and second diagnoses indicators in the new module generate for the “anemia” diagnosis.

In each of these examples, the computer system can: receive the diagnosis and corresponding set of diagnosis indicators from the provider via the builder portal; generate the diagnosis module based on the diagnosis and corresponding set of diagnosis indicators; and store the diagnosis module in the diagnostic module database.

### 13. Timeline of Diagnosis

Blocks of the method S100 recite: predicting a positive diagnosis for a diagnosis exhibited by a patient in Block S154; and appending a diagnosis timeline, representing timeseries diagnosis data exhibited by the patient over time, with the diagnosis in Block S156. In one implementation, the computer system can represent a diagnosis for a patient as a timeline of the diagnosis. For example, the computer system can implement the diagnostic module database configured to output a diagnosis timeline representing a timeseries of positive and/or negative diagnoses for the patient over a particular time period (e.g., a lifetime, an in-patient encounter).

In particular, in this implementation, the computer system can: extract a first set of patient data from a health record associated with a patient that supports a positive diagnosis of a particular diagnosis for the patient at a first time; append a diagnosis timeline with the diagnosis exhibited by the patient at the first time; extract a second set of patient data, from the health record, that supports a negative diagnosis of the particular diagnosis for the patient at a second time (e.g., a current time), succeeding the first time, during an encounter; and append the diagnosis timeline with the negative diagnosis for the patient at the second time. The computer system can then predict a positive diagnosis for the diagnosis exhibited by the patient during the encounter based on the diagnosis timeline representing the diagnosis exhibited by the patient at the first time.

In this implementation, rather than represent a diagnosis for a patient as a static diagnosis at a single point in time, the computer system can leverage the diagnostic module database to represent the diagnosis over a time period (e.g., a lifetime, a patient encounter, a particular time window), thereby representing change in the diagnosis for the patient over time. In particular, the computer system can: derive a timeseries of diagnosis data for a particular diagnosis—such as including a timeseries of “true” values and/or “false” values—based on a diagnosis module defined for the diagnosis and a timeseries of patient data extracted from the patient's electronic health record; and represent the timeseries of diagnosis data as a diagnosis timeline representing whether the patient exhibited a positive (e.g., corresponding to a “true” value) or a negative diagnosis (e.g., corresponding to a “false value) at any given time represented in the diagnosis timeline.

In one example, the computer system can generate a diagnosis timeline—for a diagnosis of anemia—based on a timeseries of diagnosis data including: a first time value—such as corresponding to a start of a patient encounter—linked to a “true” value representing a positive diagnosis of anemia for the patient at a first time corresponding to the first time value; a second time value—succeeding the first time value—linked to the “true” value representing a positive diagnosis of anemia for the patient at a second time corresponding to the second time value; and a third time value—succeeding the second time value—linked to a “false” value representing a negative diagnosis of anemia for the patient at a third time corresponding to the third time value.

### 13.1 Reporting to Provider

The computer system can then leverage the diagnosis timeline to selectively: surface key evidence—supporting a diagnosis supplied by the provider and assigned a “true” value by the diagnostic module database—to the provider within the provider portal; and/or selectively surface a predicted and/or possible diagnosis for the patient—supplemented with key evidence supporting the possible diagnosis and assigned a “true” value by the diagnostic module database—to the provider within the provider portal.

In one implementation, the computer system can: access a diagnosis timeline—generated for a particular diagnosis, in the population of diagnoses—output by the diagnostic module database; and, based on the diagnosis timeline, selectively present a particular diagnosis, in the population of diagnoses, to the provider for review, such as for possible inclusion in a note generated for a current encounter with the patient. In particular, in one example, the computer system can: access a diagnosis timeline generated for an “anemia” diagnosis for a patient and output by the diagnostic module database; identify a “positive” anemia diagnosis for the patient at a first time during a patient encounter; identify a “positive” anemia diagnosis for the patient at a second time—succeeding the first time—during the patient encounter; and identify a “negative” anemia diagnosis for the patient at a current time—succeeding the second time—during the patient encounter, such that a set of current patient data does not correspond to one or more diagnoses indicators defined in a diagnosis module, in the population of modules, corresponding to the “anemia” diagnosis. Based on the “positive” diagnosis at the first time, the “positive” diagnosis at the second time, and the “negative” diagnosis at the third time, the computer system can: generate a notification suggesting a “positive” diagnosis of anemia for this particular patient encounter, such as for including in a note generated for this particular patient encounter; populate the notification with patient data—corresponding to one or more diagnoses indicators defined in the diagnosis module corresponding to the “anemia” diagnosis—supporting the “positive” diagnosis; and present the notification to the provider within the provider portal for further review.

In this example, the computer system can therefore leverage the diagnosis timeline to interpret a “positive” diagnosis for the patient during the patient encounter—based on one or more “positive” anemia diagnoses at various timepoints throughout the patient encounter—regardless of whether the patient exhibits a “positive” diagnosis at the current time. Furthermore, in the preceding example, the computer system can append the notification with a selectable link pointing to the diagnosis timeline generated for this patient for the “anemia” diagnosis. The provider may then select this link to view the entire diagnosis timeline, rather than only view a singular “positive” diagnosis of anemia suggested for the patient encounter. The provider may therefore review the diagnosis timeline and selectively interpret a “positive” or “negative” anemia diagnosis accordingly.

Additionally or alternatively, in the preceding example, the computer system can selectively populate the notification with patient data—corresponding to one or more diagnoses indicators defined in the diagnosis module corresponding to the “anemia” diagnosis—supporting the “positive” diagnosis and corresponding to a highest likelihood of a “positive” diagnosis of anemia. In particular, in this example, the computer system can: characterize a first confidence score—representing confidence in the “positive” diagnosis for anemia at the first time—based on a first subset of patient data (e.g., corresponding to the first time) and the set of diagnosis indicators defined in the anemia diagnosis module; characterize a second confidence score—representing confidence in the “positive” diagnosis for anemia at the second time—based on a second subset of patient data (e.g., corresponding to the second time) and the set of diagnosis indicators defined in the anemia diagnosis module; and, in response to the second confidence score exceeding the first confidence score, populate the notification—suggesting the “positive” diagnosis for anemia—with the second subset of patient data in support of the “positive” diagnosis. The computer system can therefore surface the most relevant patient data—configured to support the “positive” diagnosis—to the provider, thereby enabling the provider to accept the suggested “positive” diagnosis for anemia with greater confidence.

### 13.2 Learning Progression of Diagnoses

In one variation, the computer system can leverage a population of diagnoses timelines—such as generated for a population of patients over a test period—to derive insights related to progression of a diagnosis over time. The computer system can then leverage these insights to selectively suggest new diagnoses indicators for a particular diagnosis—for including in a diagnosis module, in the diagnostic module database, associated with the particular diagnosis—associated with changes in patient data over time represented in the population of diagnoses timelines. In one implementation, the computer system can implement artificial intelligence, machine learning, regression, deep learning, a convolutional neural network, or other analysis techniques to derive correlations between changes in different types of patient data (e.g., various diagnosis indicators) over time, changes in diagnoses over time (e.g., positive or negative), co-occurrence of various diagnoses over time, etc. In particular, in one example, the computer system can leverage the population of diagnoses timelines to derive a correlation between presence of a first diagnosis at a first time and presence of a second diagnosis at a second time succeeding the first time.

Additionally or alternatively, in this variation, the computer system can leverage a population of diagnoses timelines derived for a population of patients—in combination with timeseries treatment data generated for this population of patients over a concurrent time period—to derive insights related to progression of diagnoses responsive to implementation of various treatment pathways. Similarly, in this variation, the computer system can implement artificial intelligence, machine learning, regression, deep learning, a convolutional neural network, or other analysis techniques to derive correlations between administration of various treatment pathways—such as individually and/or in combination—and changes in different types of patient data (e.g., various diagnosis indicators) over time, changes in diagnoses over time (e.g., positive or negative), co-occurrence of various diagnoses over time, etc.

### 14. Retroactive Diagnosis Prediction

In one variation, as shown in FIG. 3, in response to confirmation (e.g., approval) and storage of a new diagnosis module in the diagnostic module database, the computer system can retroactively populate the new diagnosis module with historical patient data to identify a missed diagnosis for the patient. In particular, in this variation, in response to receiving confirmation of a diagnosis module (i.e., a new diagnosis module) for a particular diagnosis, the computer system can: access the population of health records; and scan a health record, in the population of health records, associated with a patient for units of patient data supporting the diagnosis. The computer system can then, in response to extracting a set of patient data, from the health record, supporting the diagnosis for the patient, append a diagnosis timeline, associated with the patient, with the diagnosis exhibited by the second patient.

Later, during an encounter between the patient and a provider, the computer system can: access the diagnosis timeline representing the diagnosis exhibited by the patient at an initial time preceding the encounter; generate a notification indicating applicability of a new diagnosis for the patient and including a link configured to trigger rendering of a window loaded with the diagnosis timeline for review by the provider; and serve the notification to the provider (e.g., via the provider portal). Thus, the computer system can identify and surface previously missed diagnoses by applying newly approved diagnostic criteria to historical patient records.

### 15. Standardizing Historical Provider Notes

In one variation, as shown in FIG. 5, Block S180 of the method S100 recites accessing an unstructured provider note generated by a first provider for a first encounter, preceding a second encounter, between a patient and a first provider. In this variation, the computer system can: access historical provider notes (or “unstructured provider notes”)—generated for previous encounters between a patient and a provider—in varying formats; and standardize these unstructured provider notes by interpreting contextual signals (e.g., metadata, language signals, or structural elements) contained in these unstructured provider notes to map patient data to standardized medical codes (e.g., ICD-10, LOINC) or predefined note categories (e.g., clinical progress notes, diagnostic summaries, or treatment plans).

For example, the computer system can access unstructured provider notes generated by providers: in different specialties (e.g., hematologists, cardiologists, or primary care physicians); associated with various healthcare organizations employing varying electronic health record platforms; and/or associated with various healthcare institutions or practices employing different documentation standards for clinical observations, diagnoses, or procedures (e.g., narrative descriptions, structured medical coding such as SNOMED CT or ICD-10, or free-text fields for patient histories).

In this variation, for an encounter between a patient and a first provider, the computer system can: access an unstructured provider note (e.g., from the secure database), in a first format, generated during a previous encounter between the patient and a second provider; store data contained in the unstructured provider note in the health record associated with the patient; selectively transform unstructured data contained in the unstructured provider note into a target format, different from the first format; and selectively surface this transformed data—in the target format—to the second provider during the encounter. For example, the computer system can selectively surface data from the unstructured provider note in response to detecting relevant clinical context, such as overlapping diagnostic concerns, symptom progression, or shared treatment considerations between the data contained in the unstructured provider note and the clinical focus of the reviewing provider during the subsequent encounter.

### 16. Language Signal Extraction

In one variation, the computer system can: extract language signals from the unstructured provider note; and interpret related pieces of information (e.g., related information included in different fields of the unstructured provider note). For example, the unstructured provider note may include language signals corresponding to note metadata, such as: a provider identifier and a healthcare facility identifier; a timestamp corresponding to a start time of the previous encounter; and/or a note category (e.g., “progress note,” “operative note,” or “referral letter”) and a medical code corresponding to the note category (e.g., LOINC 34108-1 for progress notes). Additionally or alternatively, the unstructured provider note may include language signals corresponding to patient data, such as: a diagnosis recorded for the patient and annotated with evidence (e.g., lab results or imaging findings) supporting the diagnosis for the patient; a treatment pathway specified to treat the diagnosis (e.g., medications prescribed, or procedures performed); patient data recorded during the encounter (e.g., blood pressure, heart rate, body temperature); and/or clinical observations recorded by the provider during the previous encounter (e.g., physical exam findings or behavioral observations).

In this variation, the computer system can: access the unstructured provider note generated during the previous encounter between the patient and the provider; extract a cluster of language signals from the unstructured provider note; and aggregate related language signals, such as to identify relationships between recorded diagnoses, treatments, and observations.

### 17. Mapping Language Signals to Medical Codes

In one variation, Blocks of the method S100 recite: extracting a cluster of language signals, from an unstructured provider note, corresponding to a unit of patient data and omitting a medical code in Block S182; accessing a set of mapping rules associating language signals, corresponding to units of patient data, with the population of medical codes in Block S184; calculating a confidence score for the set of language signals corresponding to a medical code based on a correlation between the set of language signals and criteria defined for the medical code in the set of mapping rules in Block S186; and, in response to the confidence score exceeding a threshold score, storing the unit of patient data in association with the medical code in the health record associated with the patient in Block S188.

In this variation, the computer system can: isolate a set of language signals—in the cluster of language signals—corresponding to a unit of patient data and omitting corresponding medical codes; and interpret medical codes associated with these language signals. For example, the computer system can: parse a “clinical observations” section of the unstructured provider note including free-text content; and isolate a set of language signals specifying a particular diagnosis for the patient and a set of supporting evidence corresponding to the diagnosis and omitting a medical code.

The computer system can then access a set of mapping rules associating language signals—corresponding to units of patient data—with medical codes. The computer system can then, for the set of language signals corresponding to the unit of patient data, predict a confidence score for the set of language signals corresponding to a medical code—in a population of medical codes—based on a correlation between the set of language signals and criteria defined for the first medical code in the set of mapping rules. In response to the confidence score exceeding a threshold score, the computer system can: associate the medical code with the set of language signals and the unit of patient data; and store the unit of patient data in association with the medical code in a health record—in a population of health records—associated with the patient. Later, when accessing the diagnosis module (e.g., in response to receiving the diagnosis for the patient), the computer system can extract the unit of patient data from the health record associated with the patient based on the medical code. Therefore, the computer system can leverage mapping rules to interpret medical codes associated with language signals represented in an unstructured provider note.

### 18. Mapping Language Signals to Evidence Types

In one variation, the computer system can implement methods and techniques described above to interpret an evidence type, in the population of evidence types, corresponding to a unit of patient data identified in an unstructured provider note. In particular, the computer system can: access a set of mapping rules associating language signals—corresponding to units of patient data—with evidence types; predict a confidence score for a set of language signals corresponding to an evidence type based on a correlation between the set of language signals and criteria defined for the evidence type in the set of mapping rules; and, in response to the score exceeding a threshold score, store the unit of patient data in association with the evidence type in the health record associated with the patient. For example, the computer system can: isolate a set of language signals, in an unstructured provider note, corresponding to a unit of patient data, such as “hb less than 12.0 g/dL;” predict a score for “hb” corresponding to an evidence type, such as “hemoglobin;” and store the unit of patient data in association with the evidence type of “hemoglobin” in response to the score exceeding the threshold score. Later, the computer system can extract the unit of patient data from the health record associated with the patient based on the evidence type, such as in response to a query from the provider for patient data corresponding to the “hemoglobin” evidence type.

Therefore, the computer system can associate units of patient data, in unstructured provider notes generated across disparate healthcare systems, with predefined evidence types that correspond to the diagnosis indicators defined in the population of diagnosis modules. By mapping units of patient data to predefined evidence types, the computer system can standardize patient data into a format compatible with the population of diagnosis modules to facilitate rapid and comprehensive access to patient data.

### 19. Note Categorization

In one variation, the computer system can: assign a note category to the unstructured provider note; and store the unstructured provider note—in association with the first note category—in the health record associated with the patient. In particular, in this variation, the computer system can: access the unstructured provider note generated during the previous encounter between the patient and the provider; and scan the unstructured provider note for presence of language signals corresponding to a note category of the unstructured provider note.

For example, the computer system can scan the unstructured provider note for presence of language signals corresponding to a medical code linked to a note category for the unstructured provider note. In response to detecting absence of language signals corresponding to a medical code, the computer system can access a note categorizing model configured to autonomously predict a note category based on language signals extracted from the unstructured provider note. The computer system can then predict a confidence score for the unstructured provider note corresponding to a first note category based on a correlation between language signals extracted from the unstructured provider note and criteria defined for the first note category. Then, in response to the confidence score exceeding a threshold score, the computer system can: assign the first note category to the unstructured provider note; and store the unstructured provider note—in association with the first note category—in the health record associated with the patient.

Alternatively, in response to detecting presence of a medical code—corresponding to a note category—in a cluster of language signals extracted from the unstructured provider note, the computer system can: access a first set of language signals defined for the note category; and predict a confidence score for the unstructured provider note corresponding to the first note category based on a correlation between the cluster of language signals and the first set of language signals. Thus, the computer system can verify accuracy of note categories previously-assigned to unstructured provider notes.

In one example, the computer system: accesses a progress note (i.e., an unstructured provider note) generated by a primary care physician during an encounter (e.g., a routine checkup) with the patient; and extracts a cluster of language signals from the progress note, including “blood pressure: 120/80,” “routine screening,” and “no changes to treatment plan.” The computer system then, in response to detecting absence of a medical code—corresponding to a note category—in the cluster of language signals, predicts a confidence score for the progress note corresponding to a “progress note” category. In particular, the computer system can predict a confidence score of 95% based on a correlation between the cluster of language signals (e.g., “routine,” “screening,” and diagnostic metrics) and a first set of language signals defined for the “progress note” category (e.g., including terms indicating periodic evaluations and basic diagnostics). Then, in response to the confidence score exceeding a threshold score (e.g., 90%), the computer system stores the progress note—in association with the “progress note” category—in the health record associated with the patient. Thus, the computer system can categorize unstructured provider notes into predefined note categories to facilitate rapid retrieval of these provider notes during future clinical encounters.

### 20. Selective Note Surfacing Based on Medical Codes

In one variation, during an encounter between a patient and a provider, the computer system can selectively surface historical patient data—stored in the health record associated with the patient—in a target format for the provider to review during the encounter. In particular, in this variation, during the encounter, the computer system can access a diagnosis module (e.g., in response to receiving the diagnosis for the patient).

Then, for a first diagnosis indicator—defining an evidence type and a value of the evidence type that supports the diagnosis—in the set of diagnosis indicators, the computer system can: access a set of medical codes linked to the first diagnosis indicator; and scan the patient's health record for units of patient data corresponding to the set of medical codes. In response to detecting a unit of patient data—corresponding to a medical code in the set of medical codes—in the patient's health record, the computer system can: transform the unit of patient data into a unit of formatted patient data based on a target format specified for units of patient data corresponding to the evidence type; generate a notification including the unit of patient data in the target format and a prompt to review the unit of patient data for insertion into a provider note generated for the encounter; and serve the notification to the provider (e.g., via the provider portal).

Additionally, in this variation, the computer system can: retrieve the unstructured provider note including the unit of patient data; transform the unstructured provider note into a structured provider note in a target note format specified for provider notes; and append the notification with a first link configured to trigger rendering of a first window loaded with the unstructured provider note and a second link configured to trigger rendering of a second window loaded with the structured provider note. For example, the computer system can transform the unstructured provider note into a structured provider note in a target output format specified by a particular entity. In one example, the computer system can access a target format for a progress note—defined for a particular healthcare facility.

In one example, the computer system accesses an unstructured surgical note generated for a previous encounter between the patient and a first provider (e.g., the patient's cardiothoracic surgeon), the surgical note specifying: a coronary bypass procedure; and an operative report describing the procedure, findings, and recommendations. The computer system then implements methods and techniques described above: to map the cluster of language signals in the unstructured surgical note to a set of corresponding medical codes (e.g., LOINC 29749-9 [“Surgical operative note” ] and LOINC 29299-5 [“Cardiology Procedure Note” ]); and to store units of patient data in association with the set of medical codes in the patient's health record. Then, during an encounter between the patient and a second provider (e.g., the patient's cardiologist), the computer system: receives identification of a coronary artery disease diagnosis for the patient from the provider (e.g., via the provider portal); and accesses the diagnosis module for the coronary artery disease diagnosis defining a set of diagnosis indicators supporting the diagnosis. Then, for a first diagnosis indicator (e.g., “previous coronary bypass procedure”) in the set of diagnosis indicators, the computer system can: extract a set of medical codes (e.g., including prior procedures documented using LOINC codes 29749-9 and 29299-5) associated with the first diagnosis indicator; and scan the patient's health record for units of patient data corresponding to the set of medical codes.

The computer system then: accesses target formats for language signals in the unstructured surgical note, such as diagnostic details, procedure descriptions, and recommendations; and transforms the cluster of language signals in the unstructured surgical note into the corresponding target formats to generate a structured provider note. The computer system can then surface the structured provider note to the primary care physician during the encounter. Thus, the computer system can present contextually relevant information about the coronary bypass procedure in a concise, standardized format, such that the provider may quickly review and understand the patient's surgical history and incorporate this information into the current diagnostic context.

### 21. Selective Note Surfacing Based on Note Category

In one variation, during an encounter between a patient and a provider, the computer system can selectively surface historically-generated provider notes in a target format for the provider to review during the encounter based on note categories of the historically-generated provider notes. In particular, in this variation, the computer system can: receive identification of a diagnosis, in a population of diagnoses, for the patient from the provider (e.g., via the provider portal); and access a diagnosis module corresponding to the diagnosis and defining a set of note categories relevant to the diagnosis. Then, for a first note category—corresponding to a first medical code—the computer system can scan the patient's health record for unstructured provider notes corresponding to the first medical code.

In response to extracting an unstructured provider note—associated with the first medical code—from the patient's health record, the computer system can: retrieve the unstructured provider note; and transform the unstructured provider note into a structured provider note in a target format specified for provider notes of the first note category. The computer system can then implement methods and techniques described above to surface the structured provider note to the provider during the encounter. Thus, the computer system can present contextually relevant information about the patient to a provider during an encounter based on categorical assignments of historical notes stored in the patient's health record.

### 22. Disclaimer

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
during a first time period, at a computer system:
initializing a first diagnosis module, in a population of diagnosis modules, for a first diagnosis in a population of diagnoses;
accessing a medical resource specifying diagnosis indicators supporting the first diagnosis;
extracting a first cluster of language signals, corresponding to the first diagnosis, from the medical resource;
isolating a first set of language signals, in the first cluster of language signals, defining explicit diagnosis criteria for the first diagnosis;
transforming the first set of language signals into an explicit diagnosis indicator supporting the first diagnosis;
isolating a second set of language signals, in the first cluster of language signals, implying diagnosis criteria for the first diagnosis;
interpreting an interpreted diagnosis indicator supporting the first diagnosis based on the second set of language signals; and
storing the first diagnosis module, comprising the explicit diagnosis indicator and the interpreted diagnosis indicator, in a diagnostic module database comprising the population of diagnosis modules; and
during a second time period, succeeding the first time period, during a first encounter between a first patient and a first provider:
receiving the first diagnosis for the first patient from the first provider via a provider portal executing on a computing device accessed by the first provider;
accessing the first diagnosis module, in the population of diagnosis modules of the diagnostic module database, defining the explicit diagnosis indicator and the interpreted diagnosis indicator;
scanning a first health record, in a population of health records stored in an electronic health record database and associated with the first patient, for units of patient data corresponding to the explicit diagnosis indicator and the interpreted diagnosis indicator;
extracting a first set of patient data:
from the first health record;
corresponding to the explicit diagnosis indicator; and
supporting the first diagnosis for the first patient;
in response to correspondence between the first set of patient data and the first diagnosis module:
generating a first notification comprising the first set of patient data and a first prompt to review the first set of patient data and the first diagnosis for insertion into a provider note generated for the first encounter; and
serving the first notification to the first provider via the provider portal.

2. The method of claim 1:
wherein interpreting the interpreted diagnosis indicator comprises:
isolating a first subset of language signals, in the second set of language signals, comprising phrases referencing clinical observations of the interpreted diagnosis indicator;
matching the first subset of language signals to an evidence type, in a population of evidence types, based on correspondence between the first subset of language signals and criteria defined for the evidence type;
isolating a second subset of language signals, in the second set of language signals, comprising values proximal the evidence type in the medical resource;
interpreting a value of the evidence type, implied by the second subset of language signals, that indicates applicability of the first diagnosis to patients;
isolating a third subset of language signals, in the second set of language signals, that imply timing of the evidence type; and
interpreting a time window of the evidence type implied by the third subset of language signals;
wherein extracting the first set of patient data from the first health record comprises extracting a first unit of patient data from the first health record; and
wherein generating the first notification comprises generating the first notification to insert the first diagnosis into the provider note in response to:
correspondence between a first language signal in the first unit of patient data and the evidence type;
correspondence between a second language signal in the first unit of patient data and the value; and
correspondence between a third language signal in the first unit of patient data and the time window.

3. The method of claim 1:
wherein interpreting the interpreted diagnosis indicator comprises interpreting the interpreted diagnosis indicator defining a second evidence type, in a population of evidence types, based on the second set of language signals explicitly supporting the second evidence type;
wherein storing the first diagnosis module in the diagnostic module database comprises:
estimating a first range of values of the second evidence type implied by the second set of language signals;
calculating a confidence score for the first range of values of the second evidence type; and
in response to the confidence score falling below a threshold confidence score:
discarding the first range of values; and
writing the interpreted diagnosis indicator, defining the second evidence type and excluding a representative range of values, to the first diagnosis module;
wherein extracting the first set of patient data from the first health record comprises extracting the first set of patient data comprising a first unit of patient data satisfying a first value of the explicit diagnosis indicator that indicates applicability of the first diagnosis; and
further comprising, during the second time period:
in response to the first unit of patient data supporting the first diagnosis for the first patient and in response to absence of the representative range of values of the interpreted diagnosis indicator in the first diagnosis module:
generating a second notification to select the representative range of values of the second evidence type;
serving the second notification to the first provider via the provider portal;
receiving a second range of values from the first provider via the provider portal; and
appending the second range of values of the interpreted diagnosis indicator to the first diagnosis module.

4. The method of claim 1:
wherein interpreting the interpreted diagnosis indicator comprises interpreting the interpreted diagnosis indicator defining a second evidence type, in a population of evidence types, based on the second set of language signals explicitly supporting the second evidence type;
wherein storing the first diagnosis module in the diagnostic module database comprises:
estimating a first range of values of the second evidence type implied by the second set of language signals;
calculating a confidence score for the first range of values of the second evidence type; and
writing the interpreted diagnosis indicator, defining the second evidence type and associated with the first range of values and the confidence score, to the first diagnosis module; and
further comprising, during the second time period:
in response to the first unit of patient data supporting the first diagnosis for the first patient and in response to the confidence score for the interpreted diagnosis indicator falling below a threshold confidence score:
generating a second notification to select a representative range of values of the second evidence type;
serving the second notification to the first provider via the provider portal;
receiving a second range of values from the first provider via the provider portal; and
updating the first range of values, in the first diagnosis module, according to the second range of values.

5. The method of claim 1:
wherein interpreting the interpreted diagnosis indicator comprises interpreting the interpreted diagnosis indicator defining a second evidence type in a population of evidence types;
wherein storing the first diagnosis module in the diagnostic module database comprises:
estimating a second range of values of the second evidence type implied by the second set of language signals; and
writing the interpreted diagnosis indicator, defining the second evidence type and the second range of values, to the first diagnosis module; and
further comprising, during the second time period:
in response to receiving confirmation of the first diagnosis for the first patient from the first provider via the provider portal:
extracting a second value of the second evidence type from the first health record; and
updating the second range of values, in the first diagnosis module, to contain the second value.

6. The method of claim 1:
wherein initializing the first diagnosis module comprises initializing the first diagnosis module associated with a first clinical discipline related to the first diagnosis;
wherein storing the first diagnosis module in the diagnostic module database comprises:
estimating a first range of values of the interpreted diagnosis indicator implied by the second set of language signals; and
writing the interpreted diagnosis indicator, defining the first range of values, to the first diagnosis module; and
further comprising, during the second time period:
accessing a clinical discipline of the first provider;
in response to the clinical discipline of the first provider matching the first clinical discipline of the first diagnosis module:
generating a second notification to review the interpreted diagnosis indicator for insertion into the first diagnosis module; and
serving the second notification to the first provider via the provider portal;
receiving a second range of values, different from the first range of values, from the first provider via the provider portal; and
updating the first range of values, in the first diagnosis module, according to the second range of values.

7. The method of claim 1:
wherein transforming the first set of language signals into the explicit diagnosis indicator comprises transforming the first set of language signals into the explicit diagnosis indicator defining a first evidence type, in a population of evidence types, based on the first set of language signals explicitly supporting the first evidence type; and
further comprising, during the first time period:
retrieving a first set of medical codes, in a population of medical codes, linked to the first evidence type;
storing the first set of medical codes, linked to the explicit diagnosis indicator, in the first diagnosis module;
generating a second notification to review the explicit diagnosis indicator, defining the first evidence type, for insertion into the first diagnosis module;
serving the second notification to an operator via a module builder portal executing on a computing device accessed by the operator; and
in response to receiving selection of a second evidence type, in the population of evidence types, linked to the explicit diagnosis indicator from the operator via the module builder portal:
retrieving a second set of medical codes, in the population of medical codes, linked to the second evidence type; and
storing the second set of medical codes, linked to the explicit diagnosis indicator, in the first diagnosis module.

8. The method of claim 1:
wherein interpreting the interpreted diagnosis indicator comprises:
interpreting the interpreted diagnosis indicator defining a second evidence type, in a population of evidence types, based on the second set of language signals implying the second evidence type; and
estimating a first range of values of the second evidence type implied by the second set of language signals;
further comprising, during the first time period, at the computer system:
accessing a set of health records, in the population of health records, associated with positive diagnosis of the first diagnosis;
deriving a second range of values, different from the first range of values, of the second evidence type based on values of the second evidence type represented in the set of health records; and
calculating a third range of values based on a union of the first range of values and the second range of values;
wherein storing the first diagnosis module in the diagnostic module database comprises:
writing the interpreted diagnosis indicator, defining the second evidence type and the third range of values, to the first diagnosis module;
wherein extracting the first set of patient data from the first health record comprises extracting a first unit of patient data satisfying a first value of the explicit diagnosis indicator that indicates applicability of the first diagnosis for the first patient; and
further comprising, during the second time period:
in response to the first unit of patient data supporting the first diagnosis for the first patient and in response to detecting a difference between the first range of values and the second range of values:
generating a second notification to confirm the third range of values of the second evidence type; and
serving the second notification to the first provider via the provider portal.

9. The method of claim 1:
wherein interpreting the interpreted diagnosis indicator comprises:
interpreting the interpreted diagnosis indicator specifying a second disease diagnosed in patients within a second time window implied by the second set of language signals;
further comprising, during the second time period:
in response to the interpreted diagnosis indicator, defined in the first diagnosis module, defining the second disease:
accessing a second diagnosis module, in the population of diagnosis modules of the diagnostic module database, for the second disease, defining a second set of diagnosis indicators supporting the second disease;
extracting a second set of patient data, from the first health record, corresponding to the second set of diagnosis indicators and supporting the second disease for the first patient during the second time window;
predicting the second disease for the first patient during the second time window based on the second set of patient data satisfying values of the second set of diagnosis indicators, defined in the second diagnosis module, that indicate presence of the second disease; and
appending a diagnosis timeline, representing timeseries diagnosis data exhibited by the first patient over time, with the second disease for the first patient during the second time window;
wherein generating the first notification comprises generating the first notification comprising:
the first set of patient data supporting the first diagnosis; and
the first prompt to review the first set of patient data, the first diagnosis, and the diagnosis timeline for insertion into the provider note generated for the first encounter; and
wherein serving the first notification to the first provider further comprises rendering the diagnosis timeline via the provider portal.

10. The method of claim 1:
wherein transforming the first set of language signals into the explicit diagnosis indicator comprises transforming the first set of language signals into the explicit diagnosis indicator defining a first evidence type in a population of evidence types;
wherein extracting the first set of patient data from the first health record comprises extracting the first set of patient data comprising a first unit of patient data corresponding to the first evidence type;
further comprising:
accessing an unstructured provider note generated by a third provider for a third encounter, preceding the first encounter, between the first patient and the third provider;
extracting a third set of language signals, from the unstructured provider note, corresponding to the first unit of patient data and omitting an evidence type;
accessing a set of mapping rules associating language signals, corresponding to units of patient data, with the population of evidence types;
calculating a confidence score for the third set of language signals corresponding to the first evidence type based on correspondence between the third set of language signals and criteria defined for the first evidence type in the set of mapping rules; and
in response to the confidence score exceeding a threshold confidence score, storing the first unit of patient data in association with the first evidence type in the first health record associated with the first patient;
wherein generating the first notification comprises generating the first notification comprising:
the first unit of patient data supporting the first diagnosis; and
the first prompt to review the first unit of patient data and the first diagnosis for insertion into the provider note generated for the first encounter; and
wherein serving the first notification to the first provider further comprises rendering the unstructured provider note via the provider portal.

11. The method of claim 1, further comprising:
during the first time period:
generating a second notification to review the first diagnosis module for insertion into the diagnostic module database;
serving the second notification to an operator via a module builder portal executing on a computing device accessed by the operator; and
in response to receiving confirmation of the first diagnosis module from the operator via the module builder portal:
accessing the population of health records stored in the electronic health record database;
scanning a second health record, in the population of health records and associated with a second patient, for units of patient data corresponding to the explicit diagnosis indicator and the interpreted diagnosis indicator;
extracting a second unit of patient data, from the second health record, corresponding to the interpreted diagnosis indicator;
predicting the first diagnosis for the second patient at a second time, preceding the first time period, based on the second unit of patient data satisfying a second value of the interpreted diagnosis indicator, defined in the first diagnosis module, that indicates applicability of the first diagnosis; and
appending a second diagnosis timeline, representing timeseries diagnosis data exhibited by the second patient over time, with the first diagnosis for the second patient at the second time; and
during a third time period succeeding the first time period:
receiving confirmation of a second encounter between the second patient and the first provider via the provider portal;
accessing the second diagnosis timeline representing the first diagnosis for the second patient at the second time; and
in response to predicting the first diagnosis for the second patient at the second time and in response to absence of confirmation of the first diagnosis for the second patient:
generating a second notification to confirm the first diagnosis for the second patient at the second time; and
rendering the second diagnosis timeline via the provider portal.

12. The method of claim 1:
wherein transforming the first set of language signals into the explicit diagnosis indicator comprises transforming the first set of language signals into the explicit diagnosis indicator defining:
a first evidence type in a population of evidence types; and
a first value of the first evidence type that indicates applicability of the first diagnosis for the first patient;
further comprising, at the computer system:
retrieving a first set of medical codes, in a population of medical codes, linked to the first evidence type; and
storing the first set of medical codes, linked to the explicit diagnosis indicator, in the first diagnosis module; and
wherein extracting the first set of patient data from the first health record comprises extracting a first unit of patient data:
corresponding to a first medical code in the first set of medical codes; and
satisfying the first value of the first evidence type.

13. The method of claim 12, further comprising, during the second time period:
in response to receiving selection of the first unit of patient data from the first set of patient data, for insertion into the provider note, from the first provider via the provider portal, populating the provider note with:
the first unit of patient data corresponding to the explicit diagnosis indicator; and
the first medical code linked to the first unit of patient data;
calculating a first acceptance score for the provider note based on the first diagnosis, the first unit of patient data, and the first medical code, the first acceptance score representing a likelihood of acceptance of the provider note;
in response to the first acceptance score falling below a threshold acceptance score:
accessing a medical code database comprising the population of medical codes linked to evidence types; and
interpreting a second medical code, in the population of medical codes, corresponding to the first unit of patient data based on correspondence between the first unit of patient data and criteria defined for the second medical code;
populating the provider note with the second medical code linked to the first unit of patient data;
calculating a second acceptance score for the provider note based on the first diagnosis, the first unit of patient data, the first medical code, and the second medical code; and
in response to the second acceptance score exceeding the threshold acceptance score:
generating a second notification to verify the provider note; and
serving the second notification to the first provider via the provider portal.

14. The method of claim 1:
wherein interpreting the interpreted diagnosis indicator comprises interpreting the interpreted diagnosis indicator defining a second evidence type;
wherein storing the first diagnosis module in the diagnostic module database comprises:
accessing a medical test configured to yield patient data of the second evidence type; and
writing the interpreted diagnosis indicator, defining the second evidence type and associated with the medical test, to the first diagnosis module; and
further comprising, during the second time period:
in response to absence of a second unit of patient data, in the first health record, corresponding to the second evidence type:
identifying the medical test, configured to yield patient data of the second evidence type, defined in the first diagnosis module;
generating a second notification:
indicating insufficient evidence to support the first diagnosis for the first patient; and
comprising a second prompt to prescribe the medical test to the first patient; and
serving the second notification to the first provider via the provider portal.

15. The method of claim 1, further comprising, during the first time period:
generating a second notification to review the first diagnosis module for insertion into the diagnostic module database;
serving the second notification to an operator via a module builder portal executing on a computing device accessed by the operator; and
in response to receiving confirmation of the first diagnosis module from the operator via the module builder portal, retrieving:
a first set of medical codes linked to the explicit diagnosis indicator; and
a second set of medical codes linked to the interpreted diagnosis indicator.

16. A method comprising:
during a first time period, at a computer system:
initializing a first diagnosis module, in a population of diagnosis modules, for a first diagnosis in a population of diagnoses;
accessing a medical resource specifying diagnosis indicators supporting the first diagnosis;
extracting a first cluster of language signals, corresponding to the first diagnosis, from the medical resource;
interpreting a set of diagnosis indicators supporting the first diagnosis based on the first cluster of language signals;
retrieving a set of medical codes, in a population of medical codes, linked to the set of diagnosis indicators; and
storing the first diagnosis module, comprising the set of diagnosis indicators linked to the set of medical codes, in a diagnostic module database comprising the population of diagnosis modules; and
during a second time period succeeding the first time period:
receiving confirmation of a first encounter between a first patient and a first provider via a provider portal executing on a computing device accessed by the first provider;
accessing the first diagnosis module, in the population of diagnosis modules of the diagnostic module database, defining the set of diagnosis indicators;
scanning a health record, in a population of health records stored in an electronic health record database and associated with the first patient, for units of patient data corresponding to the set of diagnosis indicators;
extracting a first set of patient data, from the health record, corresponding to the set of diagnosis indicators;
predicting the first diagnosis for the first patient during the first encounter based on correspondence between the first set of patient data and the set of diagnosis indicators, defined in the first diagnosis module, that indicate presence of the first diagnosis;
generating a first notification to review the first diagnosis for including in a provider note generated for the first encounter; and
serving the first notification to the first provider via the provider portal.

17. The method of claim 16:
further comprising:
receiving the first diagnosis for a second patient from the first provider via the provider portal during a second encounter between the second patient and the first provider; and
wherein retrieving the set of medical codes comprises:
in response to receiving the first diagnosis for the second patient from the first provider, retrieving the set of medical codes linked to the set of diagnosis indicators.

18. The method of claim 16:
wherein interpreting the set of diagnosis indicators comprises interpreting an interpreted diagnosis indicator defining a first evidence type in a population of evidence types;
wherein retrieving the set of medical codes linked to the set of diagnosis indicators comprises, for the interpreted diagnosis indicator:
retrieving a first set of medical codes, in the population of medical codes, linked to the first evidence type;
wherein storing the first diagnosis module in the diagnostic module database comprises:
storing the first diagnosis module, comprising the interpreted diagnosis indicator and the first set of medical codes linked to the interpreted diagnosis indicator, in the diagnostic module database;
further comprising:
accessing an unstructured provider note generated by a second provider for a second encounter, preceding the first encounter, between the first patient and the second provider;
identifying a set of language signals, in the unstructured provider note, corresponding to a first unit of patient data and omitting a medical code in a population of medical codes;
accessing a set of mapping rules associating language signals, corresponding to units of patient data, with medical codes;
calculating a confidence score for the set of language signals corresponding to a first medical code, in the first set of medical codes, based on correspondence between the set of language signals and criteria defined for the first medical code in the set of mapping rules; and
in response to the confidence score exceeding a threshold confidence score, storing the first unit of patient data in association with the first medical code in the health record associated with the first patient; and
wherein extracting the first set of patient data from the health record comprises extracting the first set of patient data comprising the first unit of patient data corresponding to the first medical code.

19. The method of claim 16:
wherein interpreting the set of diagnosis indicators comprises:
interpreting an interpreted diagnosis indicator defining a first evidence type based on a first set of language signals, in the first cluster of language signals, implying the first evidence type;
interpreting a first value of the first evidence type that indicates applicability of the first diagnosis to patients, based on the first set of language signals implying the first value; and
interpolating a first time window of the first evidence type based on the first set of language signals; and
wherein extracting the first set of patient data from the health record comprises extracting the first set of patient data comprising a first unit of patient data:
corresponding to the first evidence type;
satisfying the first value of the first evidence type; and
recorded for the first patient within the first time window.

20. A method comprising:
during a first time period, at a computer system:
initializing a diagnosis module, in a population of diagnosis modules, for a diagnosis in a population of diagnoses;
accessing a medical resource specifying diagnosis indicators supporting the diagnosis;
extracting a cluster of language signals, corresponding to the diagnosis, from the medical resource;
interpreting a set of diagnosis indicators supporting the diagnosis based on the cluster of language signals, each diagnosis indicator in the set of diagnosis indicators defining an evidence type and a value of the evidence type that indicates applicability of the diagnosis to patients;
for a first diagnosis indicator, in the set of diagnosis indicators, defining a first evidence type and a first value of the first evidence type:
accessing a first set of data types, in a population of defined data types, of the first evidence type;
retrieving a first set of medical codes, in a population of medical codes, linked to the first set of data types; and
storing the first diagnosis indicator, linked to the first set of medical codes, in the diagnosis module; and
storing the diagnosis module in a diagnostic module database comprising the population of diagnosis modules; and
during a second time period succeeding the first time period:
receiving confirmation of an encounter between a patient and a provider via a provider portal executing on a computing device accessed by the provider;
accessing the diagnosis module, in the population of diagnosis modules of the diagnostic module database, defining the set of diagnosis indicators;
scanning a health record, in a population of health records stored in an electronic health record database associated with the patient, for units of patient data corresponding to the set of diagnosis indicators;
extracting a first unit of patient data from the health record, the first unit of patient data corresponding to a first medical code, in the first set of medical codes, and a first data type, in the first set of data types, of the first evidence type;
predicting the diagnosis for the patient during the encounter based on the first unit of patient data satisfying the first value of the first evidence type, defined in the diagnosis module, that indicates applicability of the diagnosis;
appending a diagnosis timeline, representing timeseries diagnosis data exhibited by the patient over time, with the diagnosis;
generating a notification to review the diagnosis for including in a provider note generated for the encounter; and
serving the notification to the provider via the provider portal.
